(19) United States
(12) Patent Application Publication
(10) Pub. No.: US 2026/0224356 A1
(43) Pub. Date: Aug. 6, 2026

(54) A PROSTHESIS

(71) Applicant: Medtronic, Inc., Minneapolis, MN (US)

(72) Inventors: Christopher ESO, Santa Rosa, CA (US); Matthew BIRDSALL, Bend, OR (US)

(21) Appl. No.: 19/152,149

(22) PCT Filed: Jan. 29, 2024
(86) PCT No.: PCT/IB2024/050814

§ 371 Date: Jul. 30, 2025

Related U.S. Application Data
(60) Provisional application No. 63/483,156, filed on Feb. 3, 2023.

(51) Int. Cl. A61F 2/24

(52) U.S. Cl. CPC A61F 2/2418; A61F 2/2439; A61F 2/2436

(57) ABSTRACT

A valve prosthesis is provided having a compressed delivery configuration and an expanded, deployed configuration. The valve prosthesis has a frame with a first frame portion and a second frame portion. The second frame portion is aligned longitudinally adjacent to the first frame portion when the prosthesis is in the compressed delivery configuration, and the frame is everted such that the second frame portion is radially outward from the first frame portion when the prosthesis is in the expanded, deployed configuration.

### CROSS-REFERENCE TO RELATED APPLICATIONS

This application claims the benefit of U.S. application Ser. No. 63/483,156, filed Feb. 3, 2023, the entire content of which is incorporated herein by reference.

### FIELD

The present teachings relate to a prosthesis, for example to a valve prosthesis, to a delivery catheter system for a prosthesis, and to a method of deploying a prosthesis using a delivery catheter system.

### BACKGROUND

The human heart is a four chambered, muscular organ that provides blood circulation through the body during a cardiac cycle. The four main chambers include the right atrium and right ventricle which supplies the pulmonary circulation, and the left atrium and left ventricle which supplies oxygenated blood received from the lungs into systemic circulation. To ensure that blood flows in one direction through the heart, atrioventricular valves (tricuspid and mitral valves) are present between the junctions of the atrium and the ventricles, and semi-lunar valves (pulmonary valve and aortic valve) govern the exits of the ventricles leading to the lungs and the rest of the body. These valves contain leaflets or cusps that open and close in response to blood pressure changes caused by the contraction and relaxation of the heart chambers. The valve leaflets move apart from each other to open and allow blood to flow downstream of the valve, and coapt to close and prevent backflow or regurgitation in an upstream manner.

Diseases associated with heart valves, such as those caused by damage or a defect, can include stenosis and valvular insufficiency or regurgitation. For example, valvular stenosis causes the valve to become narrowed and calcified which can prevent blood flow to a downstream heart chamber from occurring at the proper flow rate and may cause the heart to work harder to pump the blood through the diseased valve. Valvular insufficiency or regurgitation occurs when the valve does not close completely, allowing blood to flow backwards, thereby causing the heart to be less efficient. A diseased or damaged valve, which can be congenital, age-related, drug-induced, or in some instances, caused by infection, can result in an enlarged, thickened heart that loses elasticity and efficiency. Some symptoms of heart valve diseases can include weakness, shortness of breath, dizziness, fainting, palpitations, anaemia and edema, and blood clots which can increase the likelihood of stroke or pulmonary embolism. Symptoms can often be severe enough to be debilitating and/or life threatening.

Heart valve prostheses have been developed for repair and replacement of diseased and/or damaged heart valves. Such heart valve prostheses can be percutaneously delivered and deployed at the site of the diseased heart valve through catheter-based delivery systems. Such heart valve prostheses are delivered in a radially compressed or crimped configuration so that the heart valve prosthesis can be advanced through the patient's vasculature. Once positioned at the treatment site, the heart valve prosthesis is expanded to engage tissue at the diseased heart valve region to, for instance, hold the heart valve prosthesis in position.

The present disclosure relates to improvements in a heart valve prosthesis, including a heart valve prosthesis that has a low profile for transcatheter delivery through a patient's vasculature.

### SUMMARY

A first aspect of the teachings provides a prosthesis having a compressed delivery configuration and an expanded, deployed configuration, the prosthesis comprising: a frame comprising a first frame portion and a second frame portion; and wherein the second frame portion is aligned longitudinally adjacent to the first frame portion when the prosthesis is in the compressed delivery configuration, wherein the second frame portion is configured to anchor the prosthesis when the prosthesis is in the expanded deployed configuration, and wherein when the prosthesis is in the expanded, deployed configuration the frame is everted such that the second frame portion is radially outward from the first frame portion.

In some embodiments, in the compressed delivery configuration, the first and second frame portions do not overlap along a longitudinal axis of the frame. In some embodiments, in the compressed delivery configuration, the second frame portion is longitudinally offset from the first frame portion.

In some embodiments, in the deployed configuration, the second frame portion overlaps the first frame portion along a longitudinal axis of the frame. In some embodiments, in the deployed configuration, the first and second frame portions are concentric and/or coaxial. In some embodiments, first frame portion is at least partially surrounded by the second frame portion when the prosthesis is in the expanded, deployed configuration. In some embodiments, in the expanded, deployed configuration, the first frame portion defines a length that is greater than a length of the second frame portion along a longitudinal axis of the frame.

In some embodiments, the frame comprises a plurality of cells arranged in a plurality of circumferential rows, and wherein the frame is configured such that the second frame portion comprises at least one row of cells. In some embodiments, each row comprises less than twelve cells, optionally less than ten cells, for example in the range four to ten cells, for example in the range six to nine cells. In some embodiments, each row comprises three, six, or nine cells. In some embodiments, at least one cell of the plurality of cells is substantially diamond-shaped. In some embodiments, wherein each of the plurality of cells is substantially diamond-shaped. In some embodiments, wherein the frame comprises a plurality of crowns and a plurality of struts with each crown being formed between a pair of opposing struts, and wherein the plurality of cells are defined by the plurality of crowns and the plurality of struts.

In some embodiments, the second frame portion comprises at least one retention member configured for receiving a tensioning member to control eversion and/or deployment of the frame. In some embodiments, the retention member comprises an eyelet, loop, hook, ring, slit, slot, crown, or groove. In some embodiments, the frame comprises a series of end crowns at an end of the second frame portion, and wherein one or more of the end crowns comprise a retention member.

In some embodiments, the frame is formed from a self-expanding material. In some embodiments, the self-expanding material comprises a shape-memory material, for example nitinol. In some embodiments, the self-expanding material is shape set to evert the second frame portion upon expansion of the prosthesis. In some embodiments, the second frame portion comprises at least one retention member, and wherein the self-expanding material is configured to expand radial sections of the second frame portion sequentially starting from a radial section corresponding to one group of at least one retention member and then proceeding to one or more other radial sections around a perimeter of the second frame portion.

In some embodiments, the prosthesis comprises a skirt coupled to the frame.

In some embodiments, the prosthesis is a valve prosthesis and comprises a valve body disposed within and secured to the first frame portion, the valve body being configured to prevent blood flow in one direction to convey blood flow through a central lumen of the first frame portion. In some embodiments, the valve body is positioned at least partially outside of a volume circumscribed by the second frame portion when the valve prosthesis in the expanded, deployed configuration. In some embodiments, the valve body is positioned entirely outside of a volume circumscribed by the second frame portion when the valve prosthesis in the expanded, deployed configuration. In some embodiments, the heart valve prosthesis is configured for placement within a mitral heart valve or tricuspid heart valve in situ.

In some embodiments, the prosthesis comprises one or more radiopaque or echogenic markers for aligning the prosthesis with a native annulus. In some embodiments, the one or more markers are provided on the second frame portion.

A second aspect of the teachings provides a delivery catheter system comprising: a catheter assembly comprising an elongate catheter body having a distal end portion and a tensioning member; and a prosthesis having a compressed delivery configuration and an expanded, deployed configuration, said prosthesis comprising a frame comprising a first frame portion and a second frame portion, wherein the prosthesis is positioned within the catheter body in a compressed delivery configuration such that the second frame portion is aligned longitudinally with the first frame portion, wherein the prosthesis is deployable from the catheter body so as to enable the prosthesis to move from the compressed delivery configuration to the expanded, deployed configuration, wherein the frame in the expanded, deployed configuration is everted such that the second frame portion is radially outward from the first frame portion, wherein the second frame portion is configured to anchor the prosthesis when the prosthesis is in the expanded, deployed configuration, and wherein the tensioning member is actuatable to control expansion to the expanded, deployed configuration.

In some embodiments, the tensioning member comprises a suture, pull wire, circumferential wire, or purse string.

In some embodiments, in the compressed delivery configuration, the first and second frame portions do not overlap along a longitudinal axis of the frame. In some embodiments, in the compressed delivery configuration, the second frame portion is longitudinally offset from the first frame portion.

In some embodiments, in the deployed configuration, the second frame portion overlaps the first frame portion along a longitudinal axis of the frame. In some embodiments, in the deployed configuration, the first and second frame portions are concentric and/or coaxial.

In some embodiments, the elongate catheter body comprises a first housing and a second housing, and wherein the first housing is configured to retain the first frame portion in the compressed delivery configuration and the second housing is configured to retain the second frame portion in the compressed delivery configuration.

In some embodiments, the first and second housings are configured to move independently to deploy the first and second frame portions, such that when the first housing is moved it no longer retains the first frame portion in the compressed delivery configuration, when the second housing is moved, it no longer retains the second frame portion in the compressed delivery configuration, and wherein the tensioning member is configured to control expansion and compression of the second frame portion when not retained by the second housing.

In some embodiments, the elongate catheter body comprises a first housing and a capsule defining a second housing, wherein the first housing is configured to retain the first frame portion in the compressed delivery configuration and the second housing is configured to retain the second frame portion in the compressed delivery configuration. In some embodiments, the first and second housings are configured to move in opposite directions to deploy the first and second frame portions such that when the first housing is moved in a first direction it no longer retains the first frame portion in the compressed delivery configuration and when the second housing is moved in a second direction it no longer retains the second frame portion in the compressed delivery configuration, and wherein the tensioning member is configured to control expansion and compression of the second frame portion when not retained by the second housing.

In some embodiments, the delivery catheter system comprises a balloon that is donut-shaped or lasso-shaped balloon configured and arranged to dilate the second frame portion in the expanded, deployed configuration, in use, to adjust or improve fixation, anchoring, placement, or sealing of the prosthesis.

In some embodiments, the tensioning member is configured to engage the second frame portion. In some embodiments, the second frame portion comprises at least one retention member that the tensioning member is configured to engage. In some embodiments, the retention member comprises an eyelet, loop, hook, ring, slit, slot, crown, or groove.

In some embodiments, the delivery catheter system comprises a plurality of tensioning members and a plurality of retention members, wherein each tensioning member engages one or more different retention members. In some embodiments, each of the plurality of tensioning members is independently actuatable to add or release tension. In some embodiments, the tensioning member is actuatable to allow radial sections of the second frame portion to expand sequentially around a perimeter of the second frame portion.

In some embodiments, the catheter assembly comprises an atraumatic tip positioned at or near the end portion of the catheter body.

In some embodiments, the prosthesis is a valve prosthesis and comprises a valve body disposed within and secured to the first frame portion, the valve body being configured to prevent blood flow in one direction to convey blood flow through a central lumen of the first frame portion.

In some embodiments, the prosthesis is a prosthesis according to the first aspect.

A third aspect of the teachings provides a method of deploying a valve prosthesis using a delivery catheter system comprising: obtaining a catheter assembly comprising an elongate catheter body having a distal portion and a tensioning member; obtaining a frame comprising a first frame portion and a second frame portion and having a compressed delivery configuration and an expanded, deployed configuration, a valve body disposed within and secured to the first frame portion, the valve body being configured to prevent blood flow in one direction to convey blood flow through a central lumen of the first frame portion, wherein the second frame portion is aligned longitudinally with the first frame portion when the prosthesis is in the compressed delivery configuration, and wherein the second frame portion is configured to anchor the prosthesis in a native valve annulus when the prosthesis is in the expanded, deployed configuration; positioning the valve prosthesis within the catheter body in a compressed delivery configuration; deploying the valve prosthesis from a distal portion of the catheter body so by allowing it to move from the compressed delivery configuration to the expanded, deployed configuration by everting the second frame portion, such that the second frame portion is radially outward from the first frame portion; using the tension member to control expansion of the second frame portion from the compressed delivery configuration or compression of the second frame portion from the expanded, deployed configuration; wherein deploying the valve prosthesis comprises moving a first housing configured to retain the first frame portion in the compressed delivery configuration and a second housing configured to retain the second frame portion in the compressed delivery configuration independently to deploy the first and second frame portions, such that when the first housing is moved, the first frame portion transitions from the compressed delivery configuration to the expanded, deployed configuration and when the second housing is moved, it no longer retains the second frame portion in the compressed delivery configuration.

### BRIEF DESCRIPTION OF DRAWINGS

Embodiments will now be described with reference to the accompanying drawings, in which:

FIG. 1 is a schematic illustration of a heart;

FIG. 2A is a schematic view of a delivery catheter system according to an embodiment with a valve prosthesis in a delivery configuration including an atraumatic tip;

FIG. 2B is a schematic view of a delivery catheter system according to an embodiment with a valve prosthesis in a delivery configuration including an atraumatic tip;

FIG. 2C is a schematic view of a delivery catheter system according to an embodiment with a valve prosthesis in a delivery configuration including an atraumatic tip in an extended configuration;

FIG. 3 is a schematic view of a delivery catheter system;

FIG. 4 is a schematic view of the delivery catheter system of FIG. 3 with the valve prosthesis in a partially deployed configuration;

FIG. 5 is a schematic view of the delivery catheter system of FIG. 3 with the valve prosthesis in a partially deployed configuration;

FIG. 6 is a schematic side view of the delivery catheter system of FIG. 3 with the valve prosthesis in a partially deployed configuration;

FIG. 7 is a schematic view of a valve prosthesis according to an embodiment in a deployed configuration;

FIG. 8A is a schematic view of a valve prosthesis expanding against a band representing a band of native annular tissue according to an embodiment in a deployed configuration;

FIG. 8B is a schematic view of a pre-shaped valve prosthesis according to an embodiment in a deployed configuration;

FIG. 9 is a schematic view of a valve prosthesis according to an embodiment in a deployed configuration;

FIG. 10 is a schematic view of a valve prosthesis according to an embodiment in a deployed configuration;

FIG. 11 is a schematic view of a valve prosthesis according to an embodiment in a deployed configuration;

FIG. 12 is a schematic view of a valve prosthesis according to an embodiment in a deployed configuration;

FIG. 13 is a schematic view of a balloon of a delivery catheter system according to an embodiment;

FIG. 14 is a schematic view of the balloon of FIG. 13 in combination with a valve prosthesis according to an embodiment in a deployed configuration;

FIG. 15 is a schematic view of a balloon of a delivery catheter system according to an embodiment;

FIG. 16 is a schematic view of a delivery catheter system according to an embodiment;

FIG. 17 is a schematic view of the delivery catheter system of FIG. 16 with the valve prosthesis in a delivery configuration;

FIG. 18 is a schematic view of the delivery catheter system of FIG. 16 with the valve prosthesis in a partially deployed configuration;

FIG. 19 is a schematic view of the delivery catheter system of FIG. 16;

FIG. 20 is a schematic view of the delivery catheter system of FIG. 19 with the valve prosthesis in a partially deployed configuration;

FIG. 21 is a schematic view of the delivery catheter system of FIG. 16; and

FIGS. 22 to 24 are schematic views of a delivery catheter system according to an embodiment.

### DETAILED DESCRIPTION

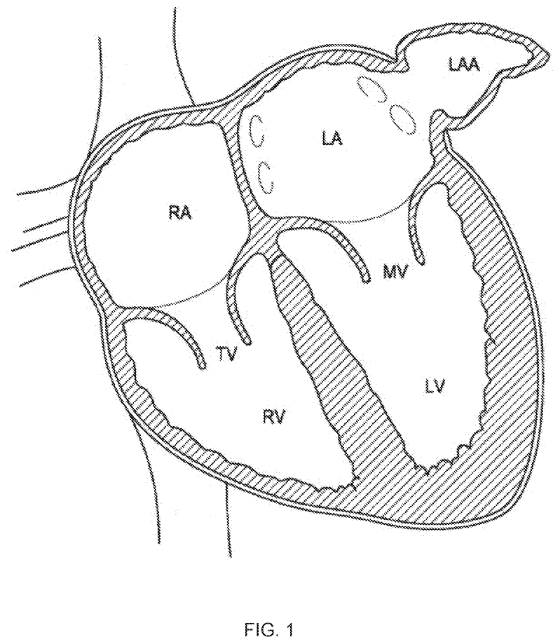

FIG. 1 is a schematic sectional illustration of a human heart. The human heart includes right and left atriums, and right and left ventricles. Disposed between the left atrium and left ventricle is the mitral valve. As left atrial pressure increases above that of left ventricle, the mitral valve opens and blood passes into the left ventricle. The mitral valve is formed from a pair of leaflets having distal edges that meet so as to close the mitral valve. Each leaflet is attached to an annular region of the heart structure known as the mitral valve annulus. Disposed between the right atrium and the right ventricle is the tricuspid valve. As right atrial pressure increases above that of right ventricle, the tricuspid valve opens and blood passes into right ventricle. The tricuspid valve is formed from three leaflets having distal edges that meet so as to close the tricuspid valve. Each leaflet is attached to the tricuspid valve annulus. In addition to mitral valve and tricuspid valve, the heart includes the aortic valve and the pulmonary valve. The aortic valve permits one-way flow of blood from the left ventricle to the aorta. The pulmonary valve permits one-way flow of blood from right ventricle to pulmonary artery. Each of the aortic valve and the pulmonary valve are formed from three leaflets having distal edges that meet so as to close the respective valve. Each leaflet is attached to an annular region of the heart structure known as the valve annulus.

Illustrated embodiments relate to a transcatheter prosthesis 12. In the exemplary illustrated embodiments, the prosthesis 12 is in the form of a heart valve prosthesis configured for placement within a mitral heart valve or a tricuspid heart valve. In alternative embodiments, the transcatheter heart valve may be configured for placement within a pulmonary, or aortic valve, or may be utilized with any transcatheter prosthesis configured for placement within a venous valve or within other body passageways where it is deemed useful.

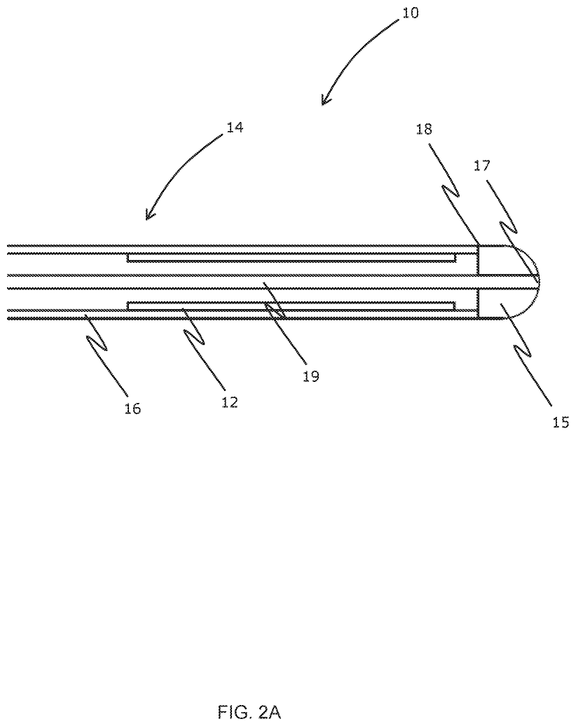

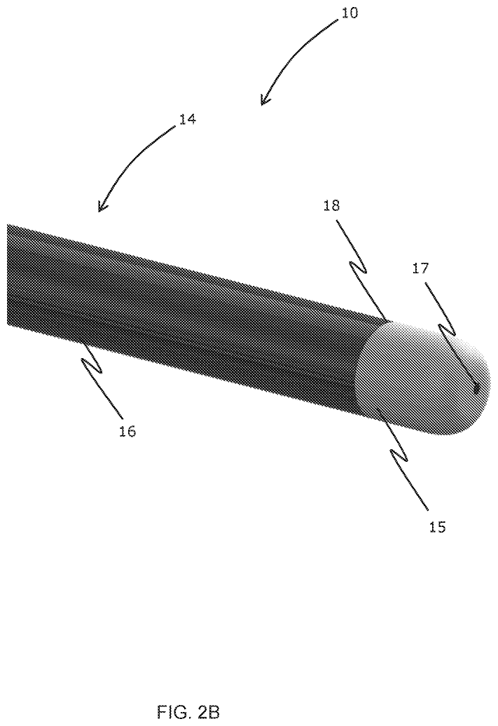

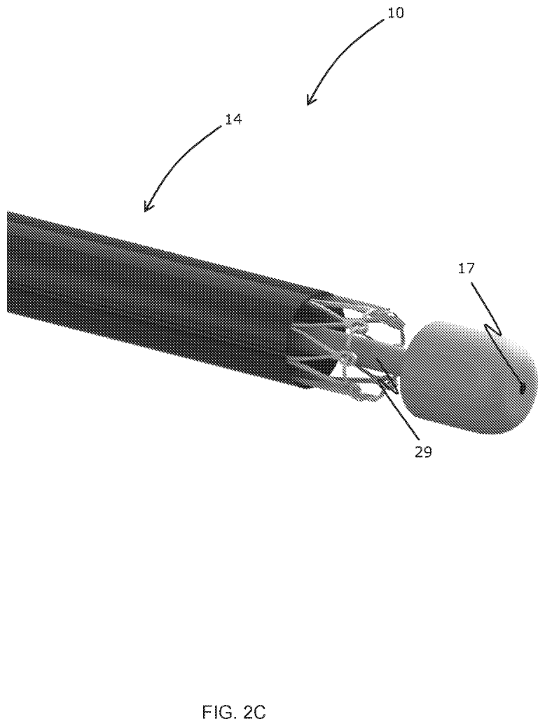

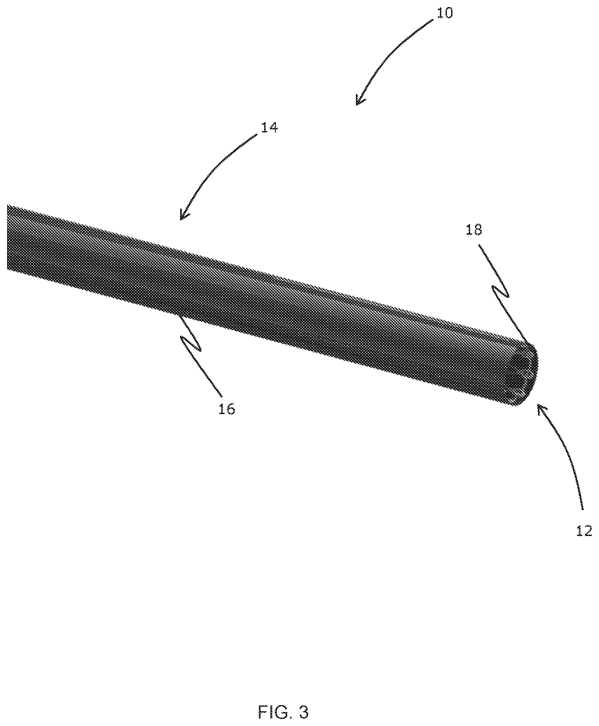

Referring to FIGS. 2A to 6, a delivery catheter system 10 is illustrated. The delivery catheter system 10 is configured for delivering and deploying a prosthesis 12 within a patient. The delivery catheter system 10 has a catheter assembly 14 including an elongate catheter body 16 having an end portion 18. The prosthesis 12 is illustrated as being positioned at least partially, for example entirely, within the catheter body 16.

In some embodiments, the catheter system 10 may include an atraumatic tip 15. The atraumatic tip 15 may be positioned at the end portion 18 of the catheter body 16. As shown in FIGS. 2B and 2C, it may have a configuration in which it is flush with end portion 18 and another configuration in which it extends away from the end portion 18. The atraumatic tip 15 may be used to facilitate the advancement of the catheter system 10 through the patient's skin and vasculature. The atraumatic tip 15 may be configured so as to prevent intravascular trauma during delivery of the catheter system 10 to the native valve annulus. In some embodiments, the atraumatic tip 15 may be a flexible curved or tapered tip (i.e. a flexible curved or tapered distal end face). In one embodiment, the atraumatic tip 15 may have a distal opening 17 for accommodating a guidewire 19. It will be understood that the taper and/or curvature of the atraumatic tip 15 may be varied depending upon the sizing/configuration as required. In some embodiments, the atraumatic tip 15 may be omitted.

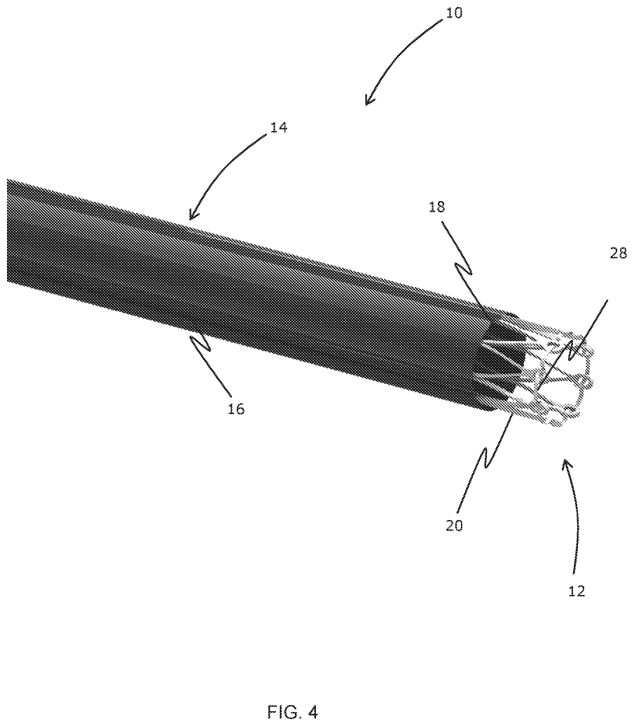

The prosthesis 12 has a compressed delivery configuration and an expanded, deployed configuration. When the prosthesis 12 is contained within the catheter body 16, as is illustrated in FIG. 2A, the prosthesis 12 is in the compressed delivery configuration. A delivery configuration may be one in which at least a part of the prosthesis 12 is in a radially compressed configuration. It will be understood that the prosthesis 12 may remain in the delivery configuration when the prosthesis 12 is partially deployed from the catheter body 16, as is illustrated in FIGS. 4-6.

Referring to FIGS. 4, 5, and 6, movement of the prosthesis 12 relative to the catheter assembly 14 enables the prosthesis 12 to move from a compressed delivery configuration to the expanded, deployed configuration.

The prosthesis 12 includes a frame 20. The frame 20 includes a first frame portion 22 and a second frame portion 24. The frame 20 is moveable between the compressed delivery configuration and the expanded, deployed configuration. The prosthesis 12 is partially deployed from the catheter assembly 14 such that the second frame portion 24 is in an expanded, deployed configuration. The first frame portion 22 is positioned within the catheter assembly 14 such that the catheter assembly 14 retains the first frame portion 22 in the compressed delivery configuration. When the prosthesis 12 is at least partially in the deployed configuration with the second frame portion 22 in a radially expanded configuration, the second frame portion 22 is configured to anchor the prosthesis 12 in the body, for example in the native valve annulus if the prosthesis is a valve prosthesis.

In some embodiments, the prosthesis 12 does not include a valve body, but may be suitable to support a valve body that may be implanted or attached within it. In other embodiments, the prosthesis 12 includes a valve body 26 disposed within and secured to the first frame portion 22. The valve body 26 may include valve leaflets, e.g., one, two or three valve leaflets. The valve leaflets are disposed to form a one-way valve, e.g., by coapting with one another, within the first frame portion 22. The valve leaflets may be formed of various materials including, but not limited to natural pericardial material such as tissue from bovine, equine or porcine origins, or synthetic materials such as polytetrafluoroethylene (PTFE), DACRON® polyester, RESILIA® tissue, pyrolytic carbon, or other biocompatible materials. With certain prosthetic leaflet materials, it may be desirable to coat one or both sides of the replacement valve leaflet with a material that will prevent or minimize overgrowth. It is further desirable that the prosthetic leaflet material is durable and not subject to stretching, deforming, or fatigue. When the prosthesis 12 is in the deployed configuration with the first frame portion 22 in a radially expanded configuration, the valve body 26 and associated leaflets can prevent blood flow in one direction to convey downstream blood flow through a central lumen of the first frame portion 22.

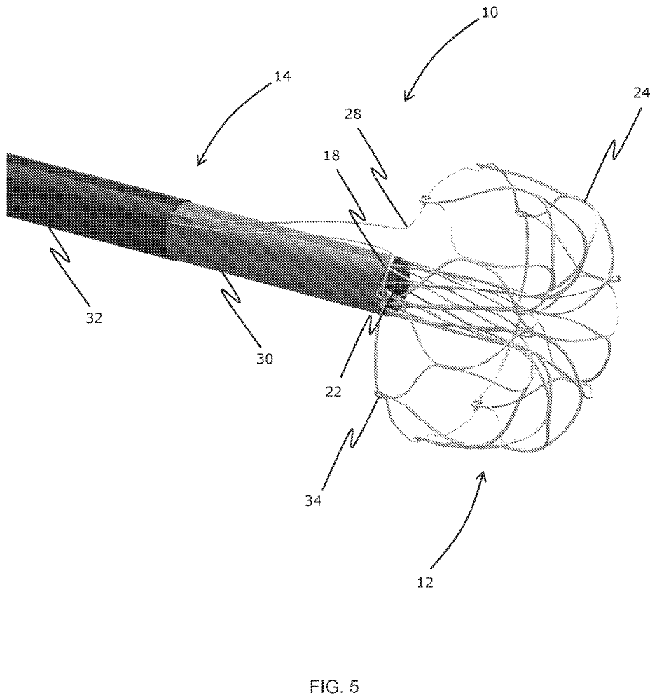

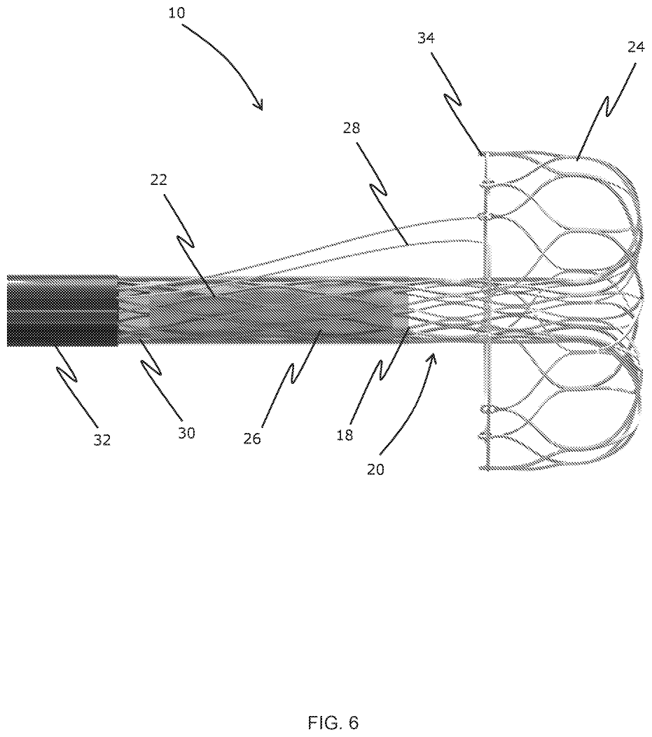

When the prosthesis 12 is in a compressed delivery configuration, as is illustrated in FIGS. 2 to 4, for example, the second frame portion 24 is aligned longitudinally with the first frame portion 22. In some embodiments, in the compressed delivery configuration, the first and second frame portions 22, 24 my not overlap along a longitudinal axis of the frame 20. This allows the prosthesis 12 to take on a compressed profile of lower diameter than if the first frame portion 22 and second frame portion 24 overlapped when compressed within the catheter assembly 14. Put another way, in some embodiments, in the compressed delivery configuration, the second frame portion 24 may be longitudinally offset from the first frame portion 22. When the second frame portion 24 of the prosthesis 12 is in a deployed or partially deployed configuration, as is illustrated in FIGS. 5 and 6, for example, the frame 20 may be everted, such that the second frame portion 24 is radially outward from the first frame portion 22. This eversion may be accomplished by, for example, shape setting a shape-memory allow, such as nitinol, to cause the eversion when released under certain temperature conditions, such as within the human body.

In some embodiments, the delivery catheter system 10 includes one or more tensioning members 28. A tensioning member 28 may be configured to control expansion of the frame 20 to an expanded, partially or fully deployed configuration. Put another way, the tensioning member 28 may be configured to control or help control eversion of all or part of the second frame portion 24 during expansion of the frame 20. The one or more tensioning members 28 may be provided in the forms of one or more sutures, one or more pull wires, one or more circumferential wires, or one or more purse strings, for example, although any suitable tensioning member or members 28 may be provided in alternative embodiments. For example, the tensioning member 28 shown in FIGS. 4-6 may be configured to engage the first frame portion 22 or the second frame portion 24, or both. In some embodiments, the second frame portion 24 may have at least one retention member 34 that the tensioning member 28 is configured to engage. The retention member 34 may be provided in the form of an eyelet, loop, hook, ring, slit, slot, crown, or groove, for example. In other embodiments, the second frame portion 24 may have a plurality of retention members 34, each having the same or varying forms, such as alternating eyelets and hooks. The retention members 34 may be oriented parallel, perpendicular, or at another angle to the longitudinal axis of the prosthesis 12 in a compressed or expanded configuration by, for example, shape setting a series of retention members 34 to take on a desired configuration upon expansion or release.

As depicted in FIGS. 5 and 6, in some embodiments, the catheter assembly 14 may include a first housing or sheath 30 and a second housing or sheath 32. In alternative embodiments, the catheter assembly 14 may include only a single sheath (i.e. the catheter body 16) In the illustrated embodiment, the first housing 30 is arranged to fit within the second housing 32. The first housing 30 may be configured and arranged to retain the first frame portion 22 in a compressed delivery configuration. The second housing 32 may be configured to retain the second frame portion 24 in a compressed delivery configuration. In some embodiments, the first and second housings 30, 32 may be configured to move independently from one another or in synchronization with one another to deploy the first and second frame portions 22, 24 independently from, or in synchronization with, one another, respectively. In some such embodiments, as one or both of the first and second housings 30, 32 are moved relative to one another, the first frame portion 22 transitions from a compressed delivery configuration to an expanded, deployed configuration. In some such embodiments, when one or more of the housings 30, 32 is or are moved sufficiently relative to one another, the second housing 32 no longer retains the second frame portion 24 in a compressed delivery configuration. In some embodiments, the tensioning member 28 may be configured to control or help control expansion, compression, and/or eversion of the second frame portion 24 when all or part of the second frame portion 24 is not retained within the second housing 32. For example, in FIG. 4, frame 20 is compressed within the catheter assembly 14. A portion of frame 20 extends outside of catheter assembly 14, but is not everting because it is has either or both of the properties that it not shape set to evert when exposed only to the point shown in FIG. 4 or tensioning member 28 is preventing it from everting by imparting radially inward tension. In FIG. 5, the first and second housings 30, 32 have moved relative to one another such that first housing 30 protrudes out of and beyond an edge of second housing 32.

In some embodiments, during deployment of the prosthesis 12 from the catheter body 16, the second housing 32 may be moved relative to the prosthesis 12 to deploy the second frame portion 24 from end portion 18 of the catheter body 16 such that the second housing 32 no longer retains the second frame portion 24 in a compressed delivery configuration. This relative movement between the prosthesis 12 and the second housing 32 may be a proximal retraction of the second housing 32. In alternative embodiments, the relative movement between the second frame portion 24 of the prosthesis 12 and the second housing may be distal movement or deployment of the prosthesis 12. In still other embodiments, the relative movement between the second frame portion 24 of the prosthesis 12 and the second housing 32 may be accomplished by a combination of distal advancement of the first housing 30 and proximal retraction of the second housing 32.

Separate from, or in combination with, expanding the second frame portion 24, the first housing 30 may be moved relative to the prosthesis 12 to deploy the first frame portion 22 from the end portion 18 of the catheter body 16 and to enable the first frame portion 22 to transition from a compressed delivery configuration to an expanded, deployed configuration. This relative movement between the first frame portion 22 of the prosthesis 12 and the first housing 30 may be a proximal retraction of the first housing 30. In alternative embodiments, the relative movement between the prosthesis 12 and the first housing 30 may be distal movement or deployment of the prosthesis 12. In still other embodiments, the relative movement between the prosthesis 12 and the first housing 30 may be accomplished by a combination of distal advancement of the prosthesis 12 and proximal retraction of the second housing 32. The tensioning member 28 may be used to prevent, slow, or otherwise control prosthesis 12 expansion throughout this process, and including, before, during, or after moving the second frame portion 24 beyond end portion 18 of catheter body 16.

The tensioning member 28 may be routed in a loop in a space between the first and second housings 30, 32 through one or more of the at least one retention member 34 of the prosthesis 12 and back along the space between the first and second housings 30, 32, or through one or more cavities, channels, ports, or tubes in the first housing 30 or second housing 32 or both housings 30, 32. The tensioning member 28 may also be routed through one or more other spaces, slits, grooves, or openings within the first or second housings 30, 32. In some embodiments, one or more tensioning members 28 are routed through a shaft connected to a distal end portion 18 to control the eversion of second frame portion 24 using, for example, tensioning members 28, extending proximally from a distal end the catheter assembly 14 rather than, or in addition to tensioning members 28 extending distally from a proximal end of catheter assembly 14 as depicted in FIG. 2C. In some embodiments, the delivery catheter system 10 may include a plurality of tensioning members 28 of one or more materials and a plurality of retention members 34. In some such embodiments, each tensioning member 28 may engage one or more different retention members 34. Each of the plurality of tensioning members 28 may be independently actuatable, or actuated in groups of one or more, to add or release tension that controls, aids, or limits expansion, compression, and/or eversion of the second frame portion 24. The plurality of tensioning members 28 may be actuated to control sequential eversion of the second frame portion 24 around a perimeter of the second frame portion 24. The plurality of tensioning members 28 may be actuated to release tension starting from a radial section corresponding to one group of at least one retention members 34, and then proceeding to one or more other radial sections around a perimeter of the second frame portion 24.

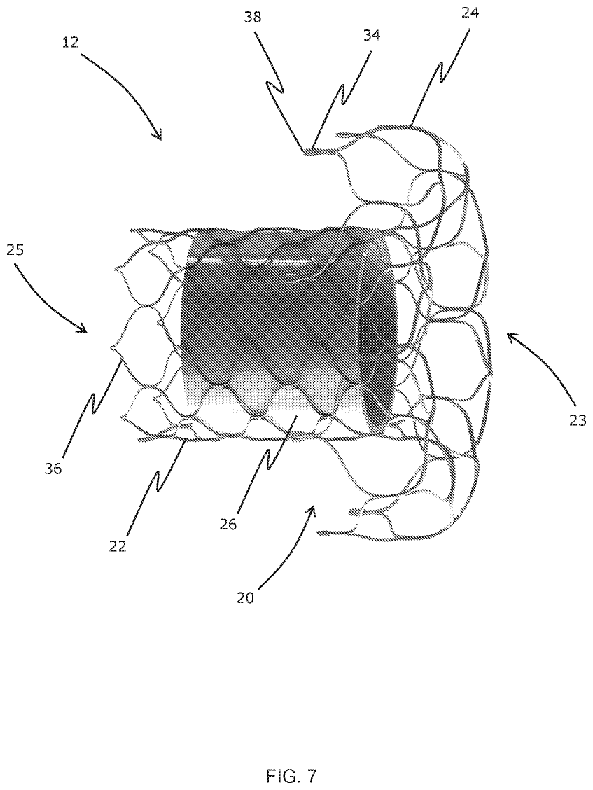

Referring to FIG. 7, the prosthesis 12 is illustrated in an expanded, deployed configuration. In the illustrated deployed configuration, the second frame portion 24 is everted, such that the second frame portion 24 extends radially outward from the first frame portion 22, and is configured to anchor the prosthesis 12 in, for example, a valve annulus, such as a native valve annulus.

In some embodiments, the second frame portion 24 may overlap the first frame portion 22 along a longitudinal axis of the frame 20 in an expanded, deployed configuration. In some embodiments, in a deployed configuration, the first and second frame portions 22, 24 may be concentric and/or coaxial. In some embodiments, the first frame portion 22 may be at least partially surrounded by the second frame portion 24, when the prosthesis 12 is in the expanded, deployed configuration. In the embodiment shown, when the prosthesis 12 is in an expanded, deployed configuration, and the first frame portion 22 defines a length that is greater than a length of the second frame portion 24 along a longitudinal axis of the frame 20. In other embodiments, the first frame portion 22 may define a length that is equal to or less than a length of the second frame portion 24 along the longitudinal axis of the frame 20.

In some embodiments, first frame portion 22 and second frame portion 24 have a generally circular elliptical cross section. In some embodiments, first frame portion 22 has a generally circular elliptical cross section and second frame portion 24 has a non-circular elliptical cross section (i.e., an ellipse having a major and minor axis of different lengths, or two different focal points). In still other embodiments, both first frame portion 22 and second frame portion 24 have non-circular elliptical cross sections. In additional embodiments, one or both of first frame portion 22 and second frame portion 24 is of a generally D-shaped cross section. Embodiments including a generally D-shaped second frame portion 24, for example, may be advantageous as a prosthetic mitral valve replacement because the native mitral valve includes a generally D-shaped annular region.

In the embodiment illustrated in FIG. 7, the valve body 26 is positioned at least partially outside of a volume circumscribed by the second frame portion 24, when the prosthesis 12 is in the expanded, deployed configuration. In alternative embodiments, the valve body 26 may be positioned entirely outside of a volume circumscribed by the second frame portion 24, when the prosthesis 12 is in the expanded, deployed configuration.

The frame 22 may include a plurality of cells 36 arranged in a plurality of circumferential rows. The rows may be arranged along the elongate length of the frame 20. In some embodiments, a row of cells 36 may include more or fewer than 12 side openings, and, for example fewer than 12 or fewer than 10 side openings. In other embodiments, a row of cells 36 may include four to ten cells 36, for example six to nine cells 36. In further alternative embodiments, a row of cells 36 may include three, six, or nine cells 36. In some embodiments, only a single row of side openings may be provided, or three or more rows of side openings may be provided. In some embodiments, each row may include three, six, or nine side openings. In some embodiments, some or all of the cell side openings are sealed by a cover, such as a skirt. In some embodiments, a skirt of fabric or tissue (not shown) may be connected to the frame 20 in a manner that controls paravalvular leakage or flow. A skirt may be attached to an inner surface, an outer surface, or an inner and an outer surface of the frame 20 around a circumference or a portion thereof. The skirt may span across or extend over a plurality of the cells 36. In some embodiments, the skirt is cut or scalloped around the edges of cells, including around crowns, such as at the edge of frame 20. In some embodiments, the skirt spans from crown to crown and is not cut or scalloped to trace cell edges or the edge of frame 20. In some embodiments, the valve body 36 may be attached to the skirt or to the skirt and frame 20. The skirt may be made from polytetrafluoroethylene (PTFE), polyethylene terephthalate (PET), or other biocompatible materials, for example. These and other variations allow for varying configurations and paravalvular sealing profiles and characteristics.

The frame 20 may include a plurality of crowns and a plurality of struts with each crown being formed between a pair or more opposing struts. Each crown may be a pointed or a curved segment or bend extending between opposing struts. The plurality of cells 36 may be defined by edges of the plurality of crowns and the plurality of struts. In an embodiment, the plurality of cells 36 may be substantially diamond, pentagonal, hexagonal, octagonal, or any other shape, such as another polygon or a circle, or any combination thereof.

As discussed above, the second frame portion 24 may have at least one retention member 34 that the tensioning member 28 is configured to engage. The retention member 34 may be provided in the form of an eyelet, loop, hook, ring, slit, slot, crown, or groove, to provide a few examples. In some embodiments, the frame 20 has a series of end crowns 38 at an end of the second frame portion 24. In such embodiments, one or more of the end crowns 38 may define one or more retention members 34.

The frame 20 may be formed from a self-expanding or balloon-expandable material, or a combination of both. The self-expanding material may be provided in the form of a shape-memory material, for example Nitinol®. The first frame portion 22 and/or second frame portion 24 may be formed at least in part, for example entirely, from the shape-memory material. The shape-memory material may be a shape-memory polymer, or a shape-memory alloy, such as Nitinol. Any portion of the frame 20 may be made from any number of suitable biocompatible materials, e.g., stainless steel, nickel titanium alloys such as Nitinol, cobalt chromium alloys such as MP35N, other alloys such as ELGILOY® (Elgin, Ill.), various polymers, pyrolytic carbon, silicone, polytetrafluoroethylene (PTFE), polyethylene terephthalate (PET), or any number of other materials or combination of materials. A suitable biocompatible material would be selected to provide the prothesis 12 to be configured to be compressed into a reduced-diameter crimped configuration for transcatheter delivery to a native valve, whereby release from a delivery catheter returns the prosthesis to an expanded, deployed configuration. In alternative embodiments, the frame (i.e., the inner and/or outer frame) may be formed from an expandable material. In such embodiments, the frame may be expanded by an expanded means, such as a balloon.

In embodiments where the second frame portion 24 is made from a self-expanding and/or shape-memory material, said material may be shape set to evert the second frame portion 24 upon expansion of the prosthesis 12 into the deployed configuration. In some such embodiments, the self-expanding and/or shape-memory material, said material may be configured to expand and evert radial sections of the second frame portion 24 sequentially. The self-expanding and/or shape-memory material may be configured to evert the second frame portion 24 starting from a radial section corresponding to one group of at least one eyelets and then proceeding to one or more other radial groups or sections around a perimeter of the second frame portion 24.

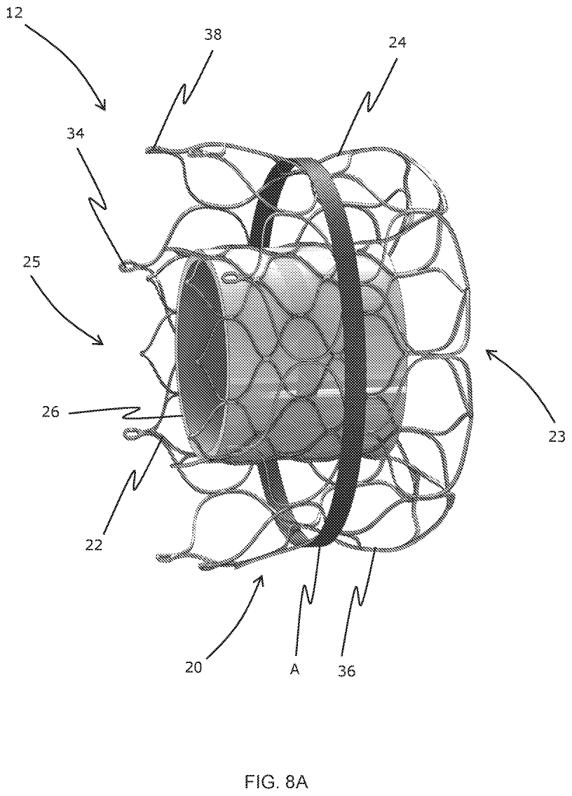

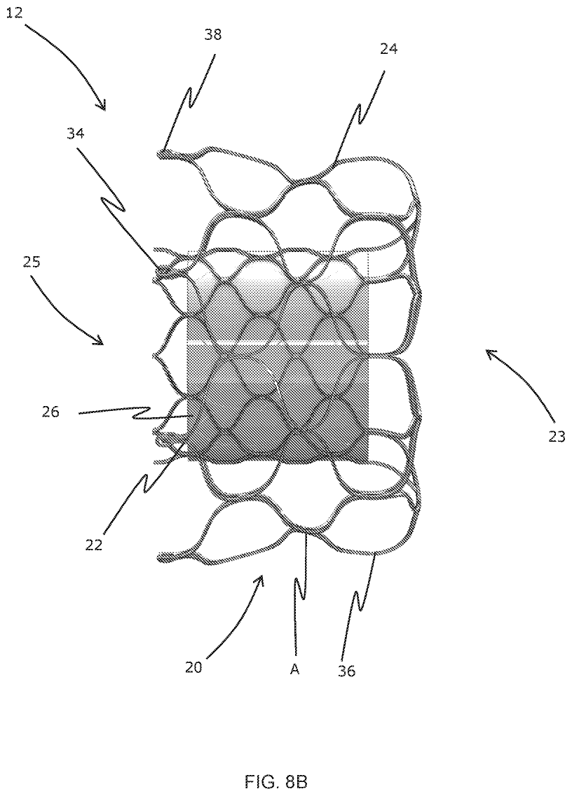

In some embodiments, for example those illustrated in FIGS. 5 to 7, the frame 20 may be configured such that the second frame portion 24 has at least one row of cells 36 thereon. In alternative embodiments, for example as illustrated in FIG. 8, the frame 20 may be configured such that the second frame portion 24 has at least two rows of cells 36 thereon, for example three rows of cells 36. In further alternative embodiments, the second frame portion 24 may include one, two, three, four, or five rows of cells 36 or any number of rows of cells to suit the application.

The prosthesis 12 illustrated in FIGS. 8A and 8B is shown schematically as being positioned within an annulus A in FIG. 8A. It will be understood that the annulus A is a schematic representation of a native valve annulus of a patient. When the prosthesis 12 is positioned within a native valve annulus A, the second frame portion 24 is deformed by the annulus A so as to conform to the annulus A. Put another way, the second frame portion 24 may be configured so as to be deformable radially inwardly for conforming to a valve annulus A. In this deformed state, the second frame portion 24 may define an hourglass shape. It will be understood that when the second frame portion 24 is deformed by the annulus A, the first frame portion 22 may not be deformed by the annulus A (for example, the first frame portion 22 may remain substantially cylindrical). In such arrangements, the second frame portion 24 may only take on this hourglass shape under an external pressure, for example from a valve annulus. The deformable second frame portion 24 may be provided on any valve prosthesis comprising an everted second frame portion, and is not limited to the valve prosthesis illustrated in FIGS. 8A and 8B.

In alternative embodiments, the second frame portion 24 may be shape-set to define an hourglass shape in the expanded, deployed configuration. Put another way, in some embodiments, the second frame portion 24 may be formed from a shape-memory material, as has been discussed above, that is configured to define an hourglass shape in the expanded, deployed configuration. In this way, the second frame portion 24 may be configured to define the hourglass shape in the absence of any external forces or resistance. As shown in FIG. 8B, the second frame portion 24 may take on this hourglass shape without an external pressure, for example resting on a benchtop. In such embodiments, the second frame portion 24 may be shape set to form a circumferential groove or channel. The groove or channel may be configured to roughly mirror a native valve annulus, in general. In such embodiments, the shape set channel or groove may acts as a locating feature to correctly align the valve prosthesis in the native valve annulus. In some embodiments, the prosthesis 12 is additionally equipped with radiopaque or echogenic markers (not shown) arranged at or around the portion meant to align with the native annulus, such as in the region of second frame portion 24 at or near annulus A in FIG. 8A. In some such embodiments, a marker is a radiopaque band of material, or one or more bands or other patterns of such material included in a skirt, attached to the inner or outer surface of second frame portion 24.

In the embodiment illustrated in FIGS. 8A and 8B, the valve body 26 is positioned at least partially within a volume circumscribed by the second frame portion 24, when the prosthesis 12 is in the expanded, deployed configuration. In some embodiments, the valve body 26 may be positioned entirely within of a volume circumscribed by the second frame portion 24, when the prosthesis 12 is in the expanded, deployed configuration.

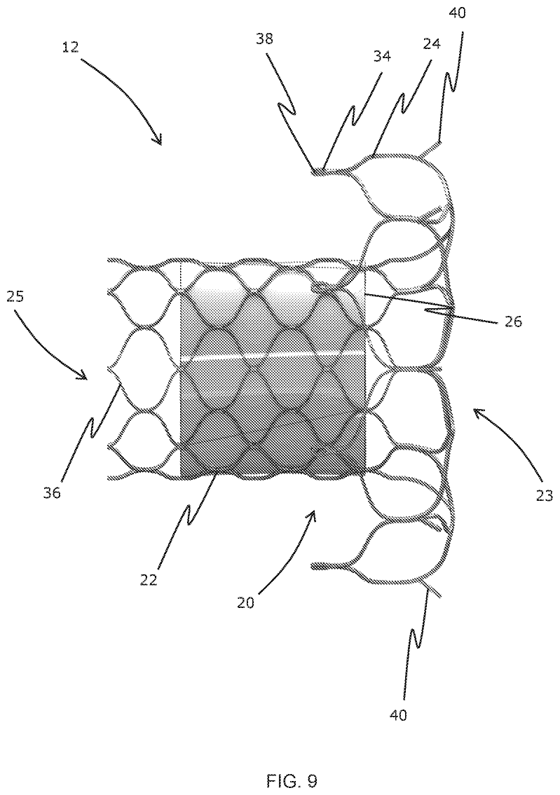

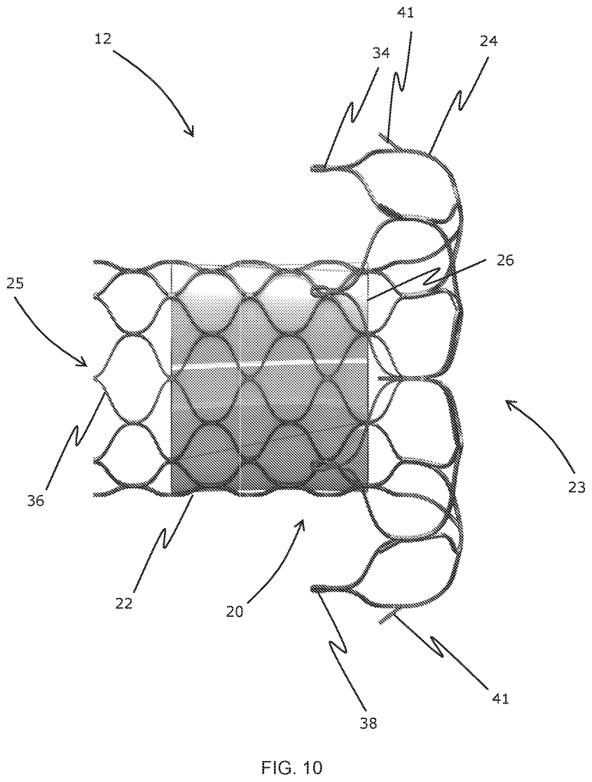

In some embodiments, the frame 20 may include one or more prongs, barbs, spikes, or other tissue fixation mechanisms 40 that extend outward from an exterior side thereof to engage heart tissue. The prongs 40 may be arranged in one or more rows or in another pattern. In the embodiment illustrated in FIG. 9, the prongs 40 are provided on the second frame portion 24. Each prong 40 extends from the second frame portion 24 in a direction toward the inflow end 23 of the prosthesis 12. In alternative embodiments, for example as illustrated in FIG. 10, the prongs 41 may extend from the second frame portion 24 in a direction toward the outflow end 25 of the prosthesis 12.

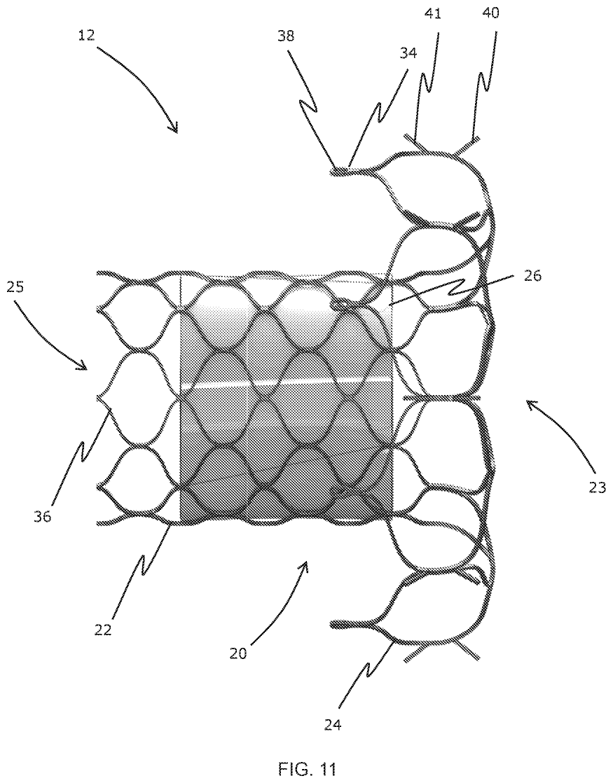

In further alternative embodiments, for example as illustrated in FIG. 11, frame 20 may be provided with a series of first prongs 40 extending from the second frame portion 24 towards the inflow end 23 and a series of second prongs 41 extending from the second frame portion 24 to the outflow end 25 of the prosthesis 12. In such arrangements, the prongs 40, 41 may be spaced apart and provided as a pair of prongs 40, 41 as is illustrated, or may be arranged adjacent to or in contact with each other to form a V-shape. In embodiments including oppositely directed prongs 40, 41, it will be understood that one of the prongs 40, 41 may be longer than the other.

In some embodiments, for example where the second frame portion 24 includes a plurality of rows of cells 36, the second frame portion 24 may include more than one row of prongs. In the illustrated embodiment, a first row of prongs is provided including a series of first prongs 40 extending from the second frame portion 24 towards the inflow end 23 and a series of second prongs 41 extending from the second frame portion 24 to the outflow end 25 of the prosthesis 12. A second row of prongs is provided on the second frame portion 24 including prongs 43 arranged in a V-shape. Although not illustrated, in some embodiments, one of the arms of the V-shaped prongs 43 may be longer than the other. In some embodiments, one or more of the prongs 40, 41, 43 may be longer than the other prongs.

It will be understood that providing a prosthesis 12 with differently directed prongs, differently sized prongs, differently located prongs, and/or different numbers of prongs can provide various options and benefits for preventing or limiting migration in varying anatomies, such as distributing securing forces along a greater distance for better securement and for greater isolation of distortions between the outer and inner portions, providing a greater surface area to land the implant in the native annulus. However, in alternative embodiments, it will be understood that in some embodiments, one or more of the prongs 40, 41, 43 may be omitted from the embodiments illustrated in FIGS. 9 to 12.

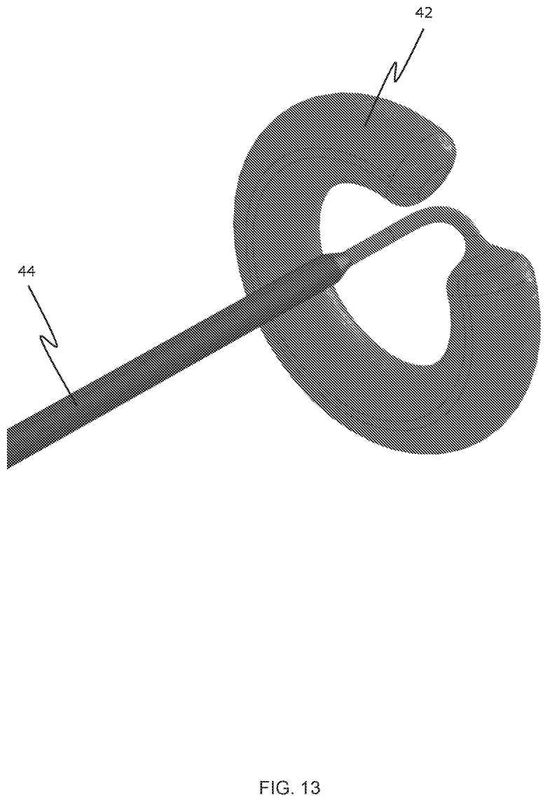

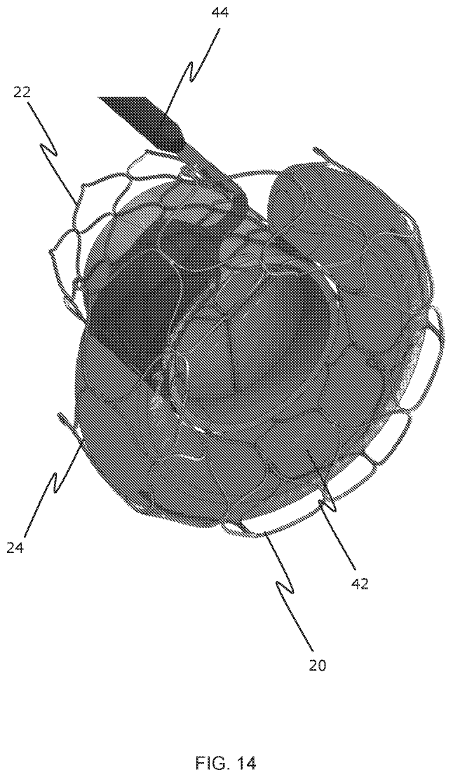

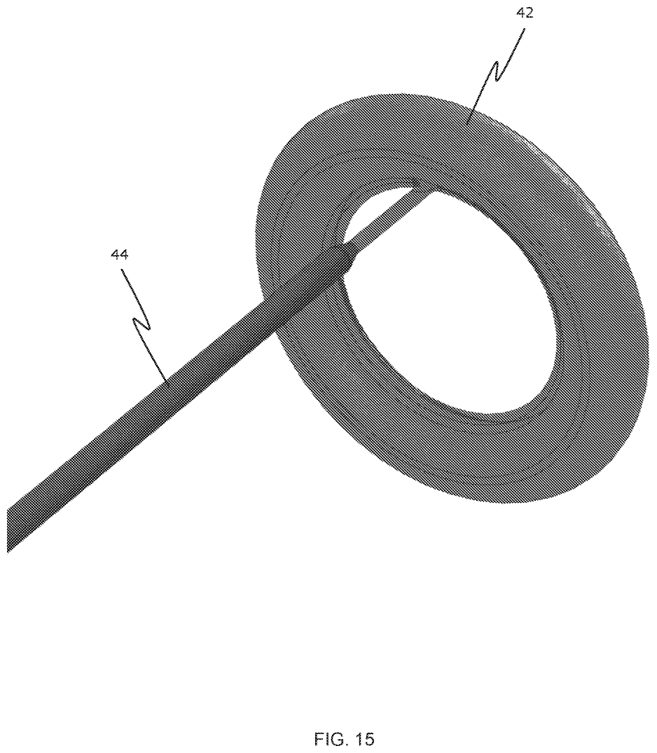

Referring to FIGS. 13 to 15, the delivery catheter system 10 may be provided with a balloon 42. The balloon 42 may be configured and arranged to dilate the second frame portion 24 in the expanded, deployed configuration, in use, to adjust or improve fixation, anchoring, placement, or sealing of the prosthesis 12 within the native valve annulus. In one embodiment, the balloon 42 may be a substantially lasso-shaped balloon, as is illustrated in FIG. 13 or 14. In another embodiment, the balloon 42 may be substantially donut-shaped as is illustrated in FIG. 15. In alternative embodiments, the balloon may be substantially C-shaped and/or may define a segment of a circle.

When the prosthesis 12 is in the expanded delivery configuration such that the second frame portion 24 is in the expanded and everted configuration, the balloon 42 is positioned in the radial spacing between the first and second frame portions 22, 24. The balloon 42 can then be inflated by filling the balloon 42 with a filter material, for example saline or a biogel. The catheter system 10 may include a delivery member 44 configured and arranged to deliver and remove a filler material into the balloon 42 to inflate and deflate the balloon 42. Control of the filling of the balloon 42 provides flexing of the second frame portion 24 to adjust or improve fixation, anchoring, placement, or sealing of the prosthesis 12 within the native valve annulus.

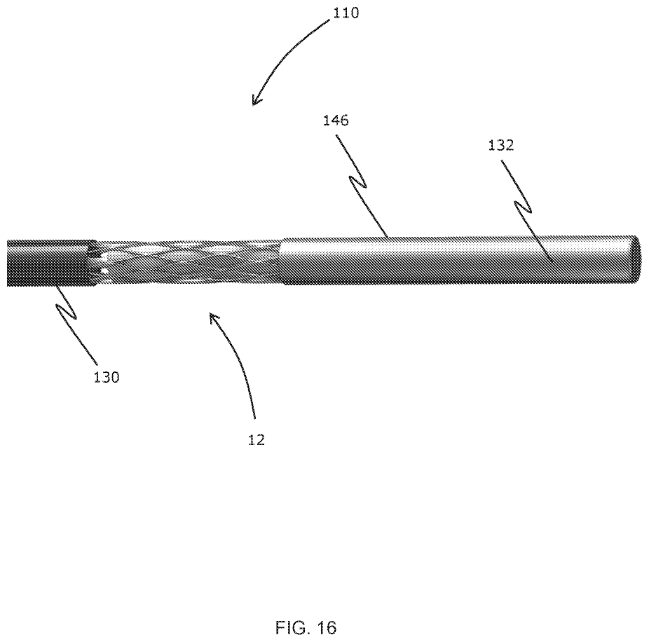

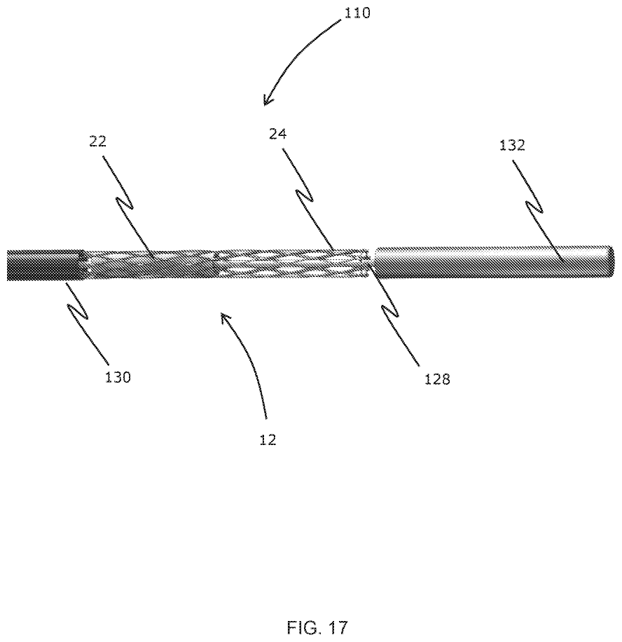

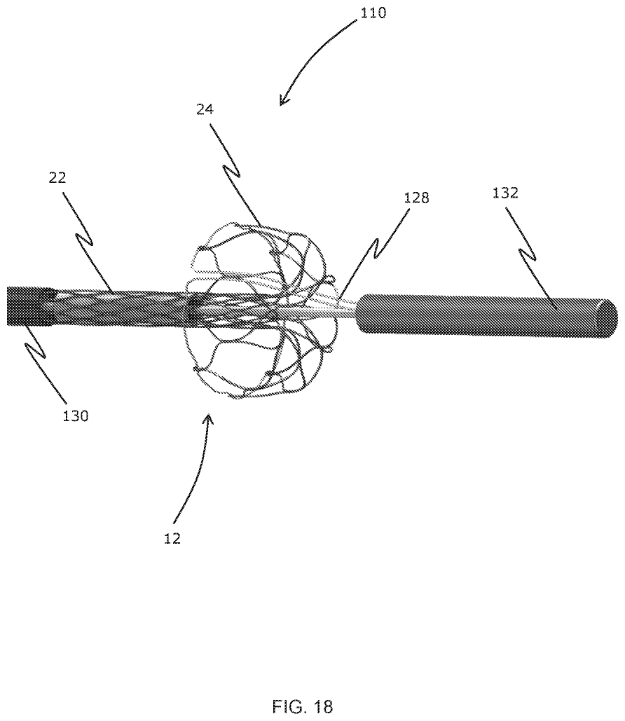

Referring to FIGS. 16 to 18, a delivery catheter system 110 is illustrated. Features common to the delivery catheter system 10 described with reference to FIGS. 2 to 6 are identified with like reference numerals with the prefix ‘1’ and only differences are discussed.

The delivery catheter system 110 is configured for delivering and deploying the prosthesis 12 within a patient. The delivery catheter system 110 has a catheter assembly 114 including an elongate catheter body 116 having a distal end portion 118. It will be understood that the prosthesis 12 is the same as has been described with reference to 2 to 8.

In this embodiment, the catheter assembly 14 includes a first housing 130 and a capsule 146. The capsule defines a second housing 132. The first housing 130 is configured to retain the first frame portion 22 in the compressed delivery configuration and the second housing 132 is configured to retain the second frame portion 24 in a compressed, delivery configuration.

The first and second housings 130, 132 may be configured to move in opposite directions to deploy the first and second frame portions 22, 24. In such embodiments, when the first housing 130 is moved in a first direction, the first frame portion 22 transitions from a compressed delivery configuration to an expanded, deployed configuration. In such embodiments, when the second housing 132 is moved in a second direction it no longer retains the second frame portion 24 in the compressed delivery configuration. In a similar manner to what has been described with reference to FIGS. 5 and 6, a tensioning member 128 may be provided to control expansion and compression of the second frame portion 24 when not retained by the second housing 132.

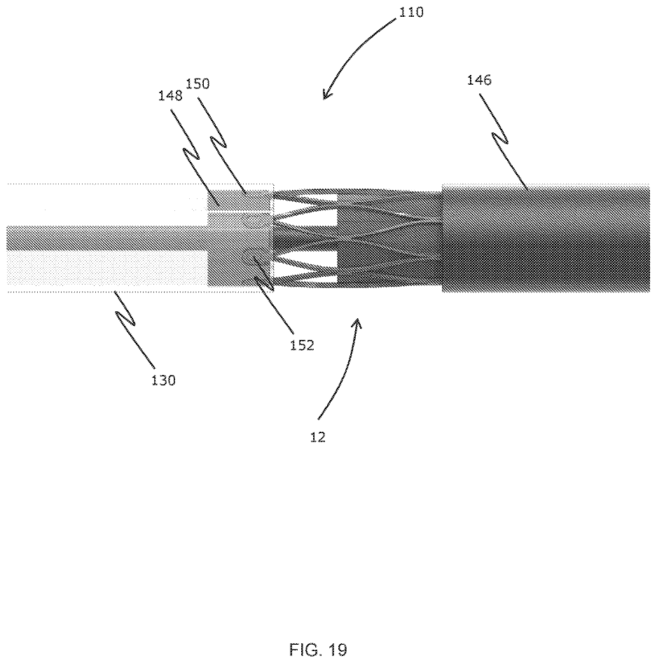

Referring to FIGS. 19 to 21, in some embodiments, the first frame portion 22 may be mounted to or connected to a mounting body 148 within the first housing 130. Put another way, the first frame portion 22 may be seated on the mounting body 148 when the prosthesis 12 is in the compressed, delivery configuration. In this embodiment, the first frame portion 22 may include one or more retention members 150. The retention member 150 may be provided in the form of an eyelet, loop, hook, ring, slit, slot, crown, or groove. Each retention member 150 may be mounted on a corresponding formation or projection of the mounting body 148.

Initially, as is illustrated in FIG. 19, the first housing 130 extends over the proximal end of the frame 20, for example a proximal end of the first frame portion 22. Put another way, initially the first housing 130 extends over and covers the retention members 150 of the first frae portion 22. In some embodiments, the first frame portion 24 may be retained in a delivery configuration by the first housing portion 130 extending over the proximal end of the frame 20.

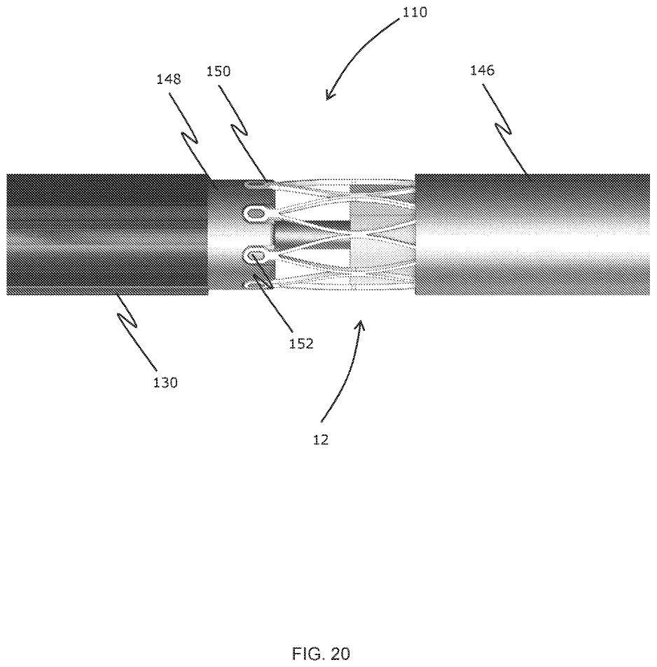

When the first housing 130 is moved in a first direction, the first frame portion 22 is deployed from the distal end portion 118 of the catheter body 116, as is illustrated in FIG. 20. Put another way, when the first housing 130 is proximally retracted the first frame portion 22 is deployed from the distal end portion 118 of the catheter body 116. In some embodiments, deployment of the first frame portion 22 from the distal end portion 118 of the catheter body 116 enables the first frame portion 22 to move from the delivery configuration to an expanded, deployed configuration.

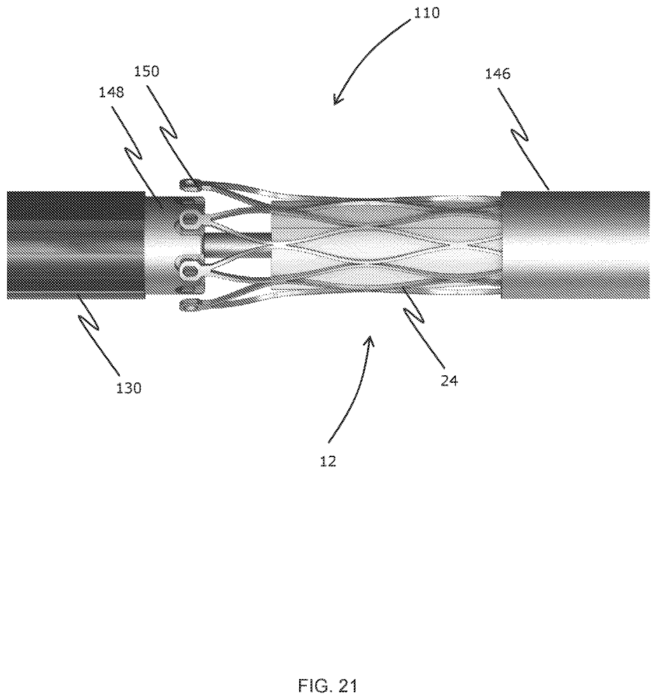

In some embodiments, for example as is illustrated in FIG. 20, the first frame portion 24 may be retained in the delivery configuration until the capsule or second housing 132 is moved distally relative to the prosthesis 12. Put another way, when the first frame portion 22 is deployed from the distal end portion 118 of the catheter body 116 the first frame portion 24 may be retained in the delivery configuration until the capsule or second housing 132 is moved distally relative to the prosthesis 12. It will be understood that the first frame portion 22 may be retained in the delivery configuration by the second housing 132 extending over the region where the first frame portion 22 is configured to expand outwardly. Distal movement of the second housing 132 relative to the prosthesis 12, as is illustrated in FIG. 21, may enable the the first frame portion 22 to move from the delivery configuration into the expanded, deployed configuration. It will be understood that the first frame portion 22 may move from the delivery configuration to the deployed configuration via one or more self-expanding or shape-memory portions of the frame 20, as has been previously discussed.

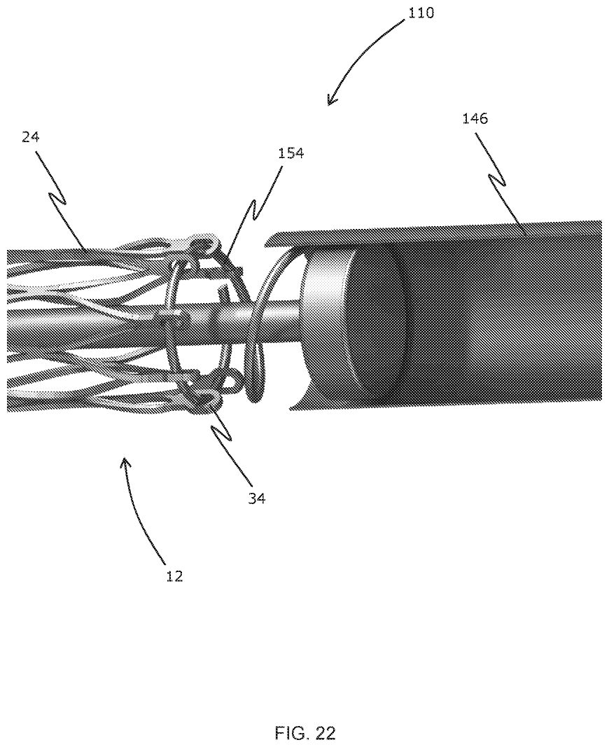

Referring to FIG. 22, an alternative tensioning member 154 of the delivery catheter system of FIGS. 16 to 21 is illustrated.

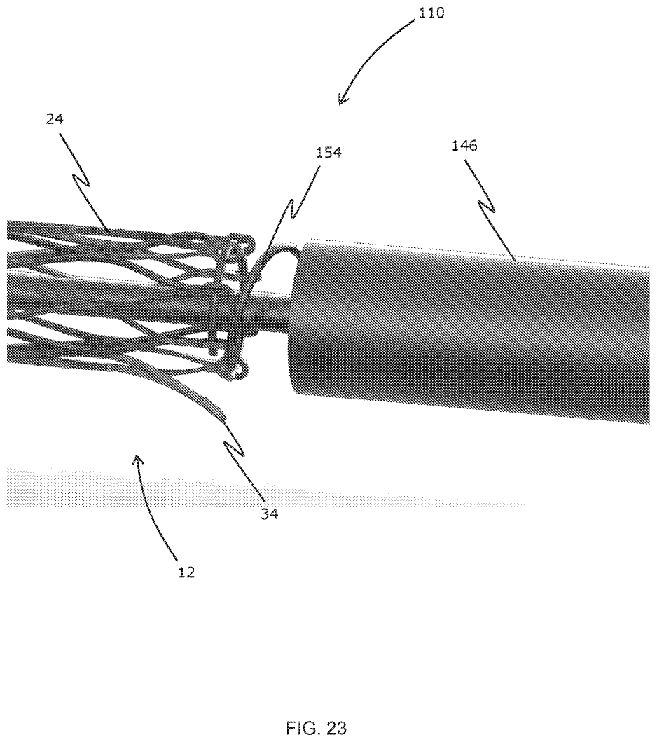

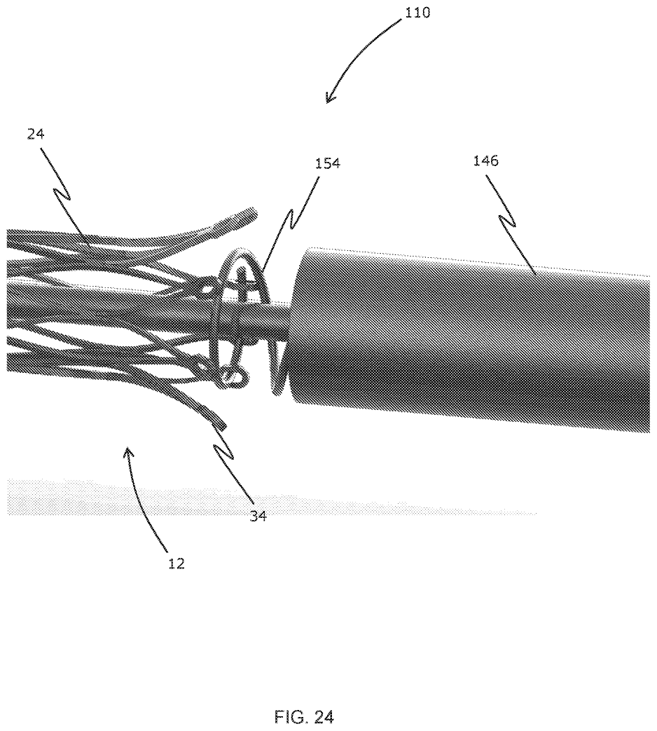

In this embodiment, the tensioning member 154 is provided in the form of a circumferential wire. The tensioning member 154 is arranged within the capsule 146. The tensioning member 154 extends through each of the retention members 34 of the prosthesis 12. In this embodiment, the tensioning member 154 retains the second frame portion 24 in the delivery configuration. The tensioning member 154 may be actuated, e.g., rotated, to release tension starting from a radial section corresponding to one retention members 34 (as is illustrated in FIG. 23), and then proceeding to the retention members 34 around a perimeter of the second frame portion 24 (as is illustrated in FIG. 24). In this way, the tensioning member 154 may be actuated to control sequential eversion of the second frame portion 24. In some embodiments, the tensioning member 154 may be used in combination with one or more of the tensioning members 28, 128 as previously discussed.

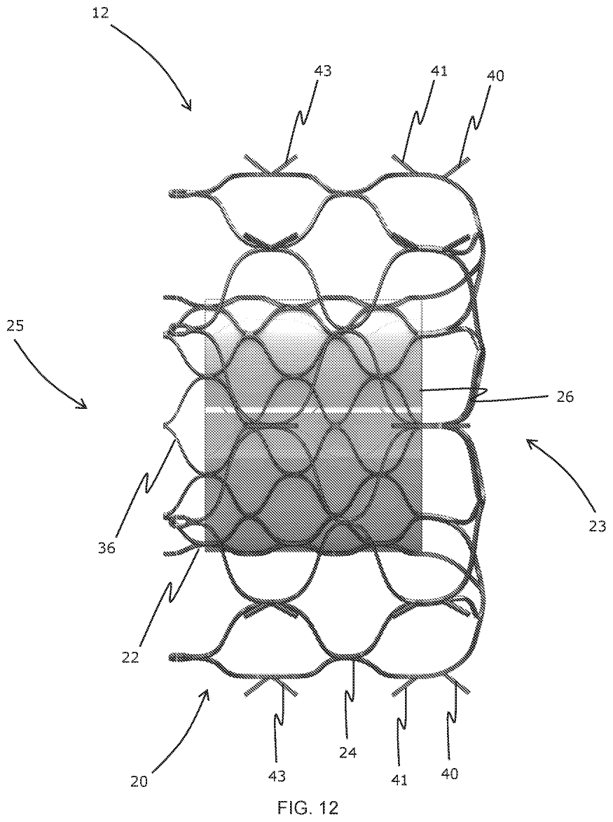

It will be understood that the delivery catheter system of FIGS. 16 to 24 may include the balloon 42 as described with reference to FIGS. 12 to 15.

A method for delivering and deploying the prosthesis 12 to a native valve of a patient will now be described.

In an embodiment, a guidewire (not shown) is advanced after having been introduced into the vasculature via a percutaneous entry point and tracked through the vasculature into a heart. Intravascular access to the right atrium RA may be achieved via a percutaneous access site to femoral venous access up to the inferior vena cava, or other known access routes. Thereafter, a guidewire is advanced through the circulatory system, eventually arriving at the heart. The guidewire is directed into the right atrium RA, and may be directed to the native tricuspid valve of the patient. In some embodiments, the guidewire may traverse the right atrium and is made to traverse, with the aid of a transseptal needle or pre-existing hole, an atrial septum, thereby entering the left atrium LA. Once the guidewire is positioned, the entry port and the atrial septum are dilated to permit entry of the catheter body 16 of the catheter system 10 into the left atrium LA. Once the guidewire is positioned, the prosthesis 12 is advanced through the catheter body 16 to the native valve and is positioned proximate the native mitral valve. It will be understood that the catheter body 16 may be positioned within the desired area of the heart via different methods or routes. For example, and not by way of limitation, another possible path would be through the radial vein into the brachial vein, through the subclavian vein, through the superior vena cava into the right atrium. Yet another possible path would be through the femoral artery into the aorta, through the aortic valve into the left ventricle, and then to the mitral valve. In another embodiment, the left ventricle LV may be accessed via a transapical approach, and the catheter body 16 may be advanced through the left ventricle LV to the mitral valve. In addition, although described with the use of a guidewire, in another embodiment hereof the catheter body 16 may access the heart without the use of a guidewire.

The method includes obtaining a catheter assembly comprising an elongate catheter body having a distal portion and a tensioning member. The method also includes obtaining a frame comprising a first frame portion and a second frame portion and having a compressed delivery configuration and an expanded, deployed configuration, a valve body disposed within and secured to the first frame portion, the valve body being configured to prevent blood flow in one direction to convey blood flow through a central lumen of the first frame portion, wherein the second frame portion is aligned longitudinally with the first frame portion when the prosthesis is in the compressed delivery configuration, and wherein the second frame portion is configured to anchor the prosthesis in a native valve annulus when the prosthesis is in the expanded, deployed configuration. The valve prosthesis is positioned within the catheter body in a compressed delivery configuration. The valve prosthesis is deployed from the distal portion of the catheter body so by allowing it to move from the compressed delivery configuration to the expanded, deployed configuration by everting the second frame portion, such that the second frame portion is radially outward from the first frame portion; using the tension member to control expansion of the second frame portion from the compressed delivery configuration or compression of the second frame portion from the expanded, deployed configuration. Deploying the valve prosthesis includes moving a first housing configured to retain the first frame portion in the compressed delivery configuration and a second housing configured to retain the second frame portion in the compressed delivery configuration independently to deploy the first and second frame portions, such that when the first housing is moved, the first frame portion transitions from the compressed delivery configuration to the expanded, deployed configuration and when the second housing is moved, it no longer retains the second frame portion in the compressed delivery configuration.

Once the catheter body 16 is in place adjacent at the native valve, the prosthesis 12 is deployed from the catheter body as has been described above. The prosthesis 12 is first partially deployed from the catheter body 16 to enable the second frame portion to move from the delivery configuration to the deployed configuration to anchor the prosthesis 12 in the native valve annulus. The prosthesis 12 is then further, e.g. fully, deployed from the catheter body 16 to enable the first frame portion to move from the radially compressed configuration to the deployed, or radially expanded configuration. Once the prosthesis 12 has been deployed, the catheter body 16 is removed and prosthesis 12 is left in the native valve annulus.

US1. A prosthesis having a compressed delivery configuration and an expanded, deployed configuration, the prosthesis comprising:
a frame comprising a first frame portion and a second frame portion; and
wherein the second frame portion is aligned longitudinally adjacent to the first frame portion when the prosthesis is in the compressed delivery configuration,
wherein the second frame portion is configured to anchor the prosthesis when the prosthesis is in the expanded deployed configuration, and
wherein when the prosthesis is in the expanded, deployed configuration the frame is everted such that the second frame portion is radially outward from the first frame portion.

US2. The prosthesis according to US1, wherein, in the compressed delivery configuration, the first and second frame portions do not overlap along a longitudinal axis of the frame.

US3. The prosthesis according to US1, wherein, in the compressed delivery configuration, the second frame portion is longitudinally offset from the first frame portion.

US4. The prosthesis according to US1, wherein, in the deployed configuration, the second frame portion overlaps the first frame portion along a longitudinal axis of the frame.

US5. The prosthesis according to US1, wherein, in the deployed configuration, the first and second frame portions are concentric and/or coaxial.

US6. The prosthesis according to US1, wherein first frame portion is at least partially surrounded by the second frame portion when the prosthesis is in the expanded, deployed configuration.

US7. The prosthesis according to US1, wherein, in the expanded, deployed configuration, the first frame portion defines a length that is greater than a length of the second frame portion along a longitudinal axis of the frame.

US8. The prosthesis according to US1, wherein the frame comprises a plurality of cells arranged in a plurality of circumferential rows, and wherein the frame is configured such that the second frame portion comprises at least one row of cells.

US9. The prosthesis according to US8, wherein each row comprises less than twelve cells, optionally less than ten cells, for example in the range four to ten cells, for example in the range six to nine cells.

US10. The prosthesis according to US8, wherein each row comprises three, six, or nine cells.

US11. The prosthesis according to US8, wherein at least one cell of the plurality of cells is substantially diamond-shaped.

US12. The prosthesis according to US11, wherein each of the plurality of cells is substantially diamond-shaped.

US13. The prosthesis according to US8, wherein the frame comprises a plurality of crowns and a plurality of struts with each crown being formed between a pair of opposing struts, and wherein the plurality of cells are defined by the plurality of crowns and the plurality of struts.

US14. The prosthesis according to US1, wherein the second frame portion comprises at least one retention member configured for receiving a tensioning member to control eversion and/or deployment of the frame.

US15. The delivery catheter system according to US14, wherein the retention member comprises an eyelet, loop, hook, ring, slit, slot, crown, or groove.

US16. The prosthesis according to US14, wherein the frame comprises a series of end crowns at an end of the second frame portion, and wherein one or more of the end crowns comprise a retention member.

US17. The prosthesis according to US1, wherein the frame is formed from a self-expanding material.

US18. The prosthesis according to US17, wherein the self-expanding material comprises a shape-memory material, for example nitinol.

US19. The prosthesis according to US17, wherein the self-expanding material is shape set to evert the second frame portion upon expansion of the prosthesis.

US20. The prosthesis according to US17, wherein the second frame portion comprises at least one retention member, and wherein the self-expanding material is configured to expand radial sections of the second frame portion sequentially starting from a radial section corresponding to one group of at least one retention member and then proceeding to one or more other radial sections around a perimeter of the second frame portion.

US21. The prosthesis according to US1, comprising a skirt coupled to the frame.

US22. The prosthesis according to US1, wherein the prosthesis is a valve prosthesis and comprises a valve body disposed within and secured to the first frame portion, the valve body being configured to prevent blood flow in one direction to convey blood flow through a central lumen of the first frame portion.

US23. The prosthesis according to US22, wherein the valve body is positioned at least partially outside of a volume circumscribed by the second frame portion when the valve prosthesis in the expanded, deployed configuration.

US24. The prosthesis according to US22, wherein the valve body is positioned entirely outside of a volume circumscribed by the second frame portion when the valve prosthesis in the expanded, deployed configuration.

US25. The prosthesis according to US22, wherein the heart valve prosthesis is configured for placement within a mitral heart valve or tricuspid heart valve in situ.

US26. The prosthesis according to US1, comprising one or more radiopaque or echogenic markers for aligning the prosthesis with a native annulus.

US27. The prosthesis according to US26, wherein the one or more markers are provided on the second frame portion.

US28. A delivery catheter system comprising:
a catheter assembly comprising an elongate catheter body having a distal end portion and a tensioning member; and
a prosthesis having a compressed delivery configuration and an expanded, deployed configuration, said prosthesis comprising a frame comprising a first frame portion and a second frame portion,
wherein the prosthesis is positioned within the catheter body in a compressed delivery configuration such that the second frame portion is aligned longitudinally with the first frame portion,
wherein the prosthesis is deployable from the catheter body so as to enable the prosthesis to move from the compressed delivery configuration to the expanded, deployed configuration,
wherein the frame in the expanded, deployed configuration is everted such that the second frame portion is radially outward from the first frame portion, wherein the second frame portion is configured to anchor the prosthesis when the prosthesis is in the expanded, deployed configuration, and
wherein the tensioning member is actuatable to control expansion to the expanded, deployed configuration.

US29. The delivery catheter system according to US28, wherein the tensioning member comprises a suture, pull wire, circumferential wire, or purse string.

US30. The delivery catheter system according to US28, wherein, in the compressed delivery configuration, the first and second frame portions do not overlap along a longitudinal axis of the frame.

US31. The delivery catheter system according to US28, wherein, in the compressed delivery configuration, the second frame portion is longitudinally offset from the first frame portion.

US32. The delivery catheter system according to US28, wherein, in the deployed configuration, the second frame portion overlaps the first frame portion along a longitudinal axis of the frame.

US33. The delivery catheter system according to US28, wherein, in the deployed configuration, the first and second frame portions are concentric and/or coaxial.

US34. The delivery catheter system according to US28, wherein the elongate catheter body comprises a first housing and a second housing, and wherein the first housing is configured to retain the first frame portion in the compressed delivery configuration and the second housing is configured to retain the second frame portion in the compressed delivery configuration.

US35. The delivery catheter system according to US34, wherein the first and second housings are configured to move independently to deploy the first and second frame portions, such that when the first housing is moved it no longer retains the first frame portion in the compressed delivery configuration, when the second housing is moved, it no longer retains the second frame portion in the compressed delivery configuration, and wherein the tensioning member is configured to control expansion and compression of the second frame portion when not retained by the second housing.

US36. The delivery catheter system according to US28, wherein the elongate catheter body comprises a first housing and a capsule defining a second housing, wherein the first housing is configured to retain the first frame portion in the compressed delivery configuration and the second housing is configured to retain the second frame portion in the compressed delivery configuration.

US37. The delivery catheter system according to US36, wherein the first and second housings are configured to move in opposite directions to deploy the first and second frame portions such that when the first housing is moved in a first direction it no longer retains the first frame portion in the compressed delivery configuration and when the second housing is moved in a second direction it no longer retains the second frame portion in the compressed delivery configuration, and wherein the tensioning member is configured to control expansion and compression of the second frame portion when not retained by the second housing.

US38. The delivery catheter system according to US28, comprising a balloon that is donut-shaped or lasso-shaped balloon configured and arranged to dilate the second frame portion in the expanded, deployed configuration, in use, to adjust or improve fixation, anchoring, placement, or sealing of the prosthesis.

US39. The delivery catheter system according to US28, wherein the tensioning member is configured to engage the second frame portion.

US40. The delivery catheter system according to US39, wherein the second frame portion comprises at least one retention member that the tensioning member is configured to engage.

US41. The delivery catheter system according to US40, wherein the retention member comprises an eyelet, loop, hook, ring, slit, slot, crown, or groove.

US42. The delivery catheter system according to US28, comprising a plurality of tensioning members and a plurality of retention members, wherein each tensioning member engages one or more different retention members.

US43. The delivery catheter system according to US42, wherein each of the plurality of tensioning members is independently actuatable to add or release tension.

US44. The delivery catheter system according to US28, wherein the tensioning member is actuatable to allow radial sections of the second frame portion to expand sequentially around a perimeter of the second frame portion.

US45. The delivery catheter system according to US28, wherein the catheter assembly comprises an atraumatic tip positioned at or near the end portion of the catheter body.

US46. The delivery catheter system according to US28, wherein the prosthesis is a valve prosthesis and comprises a valve body disposed within and secured to the first frame portion, the valve body being configured to prevent blood flow in one direction to convey blood flow through a central lumen of the first frame portion.

US47. A method of deploying a valve prosthesis using a delivery catheter system comprising:
obtaining a catheter assembly comprising an elongate catheter body having a distal portion and a tensioning member;
obtaining a frame comprising a first frame portion and a second frame portion and having a compressed delivery configuration and an expanded, deployed configuration, a valve body disposed within and secured to the first frame portion, the valve body being configured to prevent blood flow in one direction to convey blood flow through a central lumen of the first frame portion, wherein the second frame portion is aligned longitudinally with the first frame portion when the prosthesis is in the compressed delivery configuration, and wherein the second frame portion is configured to anchor the prosthesis in a native valve annulus when the prosthesis is in the expanded, deployed configuration;
positioning the valve prosthesis within the catheter body in a compressed delivery configuration;
deploying the valve prosthesis from a distal portion of the catheter body so by allowing it to move from the compressed delivery configuration to the expanded, deployed configuration by everting the second frame portion, such that the second frame portion is radially outward from the first frame portion; using the tension member to control expansion of the second frame portion from the compressed delivery configuration or compression of the second frame portion from the expanded, deployed configuration;
wherein deploying the valve prosthesis comprises moving a first housing configured to retain the first frame portion in the compressed delivery configuration and a second housing configured to retain the second frame portion in the compressed delivery configuration independently to deploy the first and second frame portions, such that when the first housing is moved, the first frame portion transitions from the compressed delivery configuration to the expanded, deployed configuration and when the second housing is moved, it no longer retains the second frame portion in the compressed delivery configuration.

EP1. A prosthesis having a compressed delivery configuration and an expanded, deployed configuration, the prosthesis comprising:
a frame comprising a first frame portion and a second frame portion; and wherein the second frame portion is aligned longitudinally adjacent to the first frame portion when the prosthesis is in the compressed delivery configuration,
wherein the second frame portion is configured to anchor the prosthesis when the prosthesis is in the expanded deployed configuration, and
wherein when the prosthesis is in the expanded, deployed configuration the frame is everted such that the second frame portion is radially outward from the first frame portion.

EP2. The prosthesis according to EP1, wherein, in the compressed delivery configuration, the first and second frame portions do not overlap along a longitudinal axis of the frame.

EP3. The prosthesis according to EP1 or EP2, wherein, in the compressed delivery configuration, the second frame portion is longitudinally offset from the first frame portion.

EP4. The prosthesis according to any of EP1-EP3, wherein, in the deployed configuration, the second frame portion overlaps the first frame portion along a longitudinal axis of the frame.

EP5. The prosthesis according to any of EP1-EP4, wherein, in the deployed configuration, the first and second frame portions are concentric and/or coaxial.

EP6. The prosthesis according to any of EP1-EP5, wherein first frame portion is at least partially surrounded by the second frame portion when the prosthesis is in the expanded, deployed configuration.

EP7. The prosthesis according to any of EP1-EP6, wherein, in the expanded, deployed configuration, the first frame portion defines a length that is greater than a length of the second frame portion along a longitudinal axis of the frame.

EP8. The prosthesis according to any of EP1-EP7, wherein the frame comprises a plurality of cells arranged in a plurality of circumferential rows, and wherein the frame is configured such that the second frame portion comprises at least one row of cells.

EP9. The prosthesis according to EP8, wherein each row comprises less than twelve cells, optionally less than ten cells, for example in the range four to ten cells, for example in the range six to nine cells.

EP10. The prosthesis according to EP8 or EP9, wherein each row comprises three, six, or nine cells.

EP11. The prosthesis according to any one of EP8 to EP10, wherein at least one cell of the plurality of cells is substantially diamond-shaped.

EP12. The prosthesis according to EP11, wherein each of the plurality of cells is substantially diamond-shaped.

EP13. The prosthesis according to any one of EP8 to EP12, wherein the frame comprises a plurality of crowns and a plurality of struts with each crown being formed between a pair of opposing struts, and wherein the plurality of cells are defined by the plurality of crowns and the plurality of struts.

EP14. The prosthesis according to any of EP1-EP13, wherein the second frame portion comprises at least one retention member configured for receiving a tensioning member to control eversion and/or deployment of the frame.

EP15. The delivery catheter system according to EP14, wherein the retention member comprises an eyelet, loop, hook, ring, slit, slot, crown, or groove.

EP16. The prosthesis according to EP14 or EP15, wherein the frame comprises a series of end crowns at an end of the second frame portion, and wherein one or more of the end crowns comprise a retention member.

EP17. The prosthesis according to any of EP1-EP16, wherein the frame is formed from a self-expanding material.

EP18. The prosthesis according to EP17, wherein the self-expanding material comprises a shape-memory material, for example nitinol.

EP19. The prosthesis according to EP17 or EP18, wherein the self-expanding material is shape set to evert the second frame portion upon expansion of the prosthesis.

EP20. The prosthesis according to any one of EP17 to EP19, wherein the second frame portion comprises at least one retention member, and wherein the self-expanding material is configured to expand radial sections of the second frame portion sequentially starting from a radial section corresponding to one group of at least one retention member and then proceeding to one or more other radial sections around a perimeter of the second frame portion.

EP21 The prosthesis according to any of EP1-EP20, comprising a skirt coupled to the frame.

EP22. The prosthesis according to any of EP1-EP21, wherein the prosthesis is a valve prosthesis and comprises a valve body disposed within and secured to the first frame portion, the valve body being configured to prevent blood flow in one direction to convey blood flow through a central lumen of the first frame portion.

EP23. The prosthesis according to EP22, wherein the valve body is positioned at least partially outside of a volume circumscribed by the second frame portion when the valve prosthesis in the expanded, deployed configuration.

EP24. The prosthesis according to EP22 or EP23, wherein the valve body is positioned entirely outside of a volume circumscribed by the second frame portion when the valve prosthesis in the expanded, deployed configuration.

EP25. The prosthesis according to any one of EP22 to EP24, wherein the heart valve prosthesis is configured for placement within a mitral heart valve or tricuspid heart valve in situ.

EP26. The prosthesis according to any of EP1-EP25, comprising one or more radiopaque or echogenic markers for aligning the prosthesis with a native annulus.

EP27. The prosthesis according to EP26, wherein the one or more markers are provided on the second frame portion.

EP28. A delivery catheter system comprising:
a catheter assembly comprising an elongate catheter body having a distal end portion and a tensioning member; and
a prosthesis having a compressed delivery configuration and an expanded, deployed configuration, said prosthesis comprising a frame comprising a first frame portion and a second frame portion,
wherein the prosthesis is positioned within the catheter body in a compressed delivery configuration such that the second frame portion is aligned longitudinally with the first frame portion,
wherein the prosthesis is deployable from the catheter body so as to enable the prosthesis to move from the compressed delivery configuration to the expanded, deployed configuration,
wherein the frame in the expanded, deployed configuration is everted such that the second frame portion is radially outward from the first frame portion, wherein the second frame portion is configured to anchor the prosthesis when the prosthesis is in the expanded, deployed configuration, and
wherein the tensioning member is actuatable to control expansion to the expanded, deployed configuration.

EP29. The delivery catheter system according to EP28, wherein the tensioning member comprises a suture, pull wire, circumferential wire, or purse string.

EP30. The delivery catheter system according to EP28 or EP29, wherein, in the compressed delivery configuration, the first and second frame portions do not overlap along a longitudinal axis of the frame.

EP31. The delivery catheter system according to any one of EP28 to EP30, wherein, in the compressed delivery configuration, the second frame portion is longitudinally offset from the first frame portion.

EP32. The delivery catheter system according to any one of EP28 to EP31, wherein, in the deployed configuration, the second frame portion overlaps the first frame portion along a longitudinal axis of the frame.

EP33. The delivery catheter system according to any one of EP28 to EP32, wherein, in the deployed configuration, the first and second frame portions are concentric and/or coaxial.

EP34. The delivery catheter system according to any one of EP28 to EP33, wherein the elongate catheter body comprises a first housing and a second housing, and wherein the first housing is configured to retain the first frame portion in the compressed delivery configuration and the second housing is configured to retain the second frame portion in the compressed delivery configuration.

EP35. The delivery catheter system according to EP34, wherein the first and second housings are configured to move independently to deploy the first and second frame portions, such that when the first housing is moved it no longer retains the first frame portion in the compressed delivery configuration, when the second housing is moved, it no longer retains the second frame portion in the compressed delivery configuration, and wherein the tensioning member is configured to control expansion and compression of the second frame portion when not retained by the second housing.

EP36. The delivery catheter system according to any one of EP28 to EP35, wherein the elongate catheter body comprises a first housing and a capsule defining a second housing, wherein the first housing is configured to retain the first frame portion in the compressed delivery configuration and the second housing is configured to retain the second frame portion in the compressed delivery configuration.

EP37. The delivery catheter system according to EP36, wherein the first and second housings are configured to move in opposite directions to deploy the first and second frame portions such that when the first housing is moved in a first direction it no longer retains the first frame portion in the compressed delivery configuration and when the second housing is moved in a second direction it no longer retains the second frame portion in the compressed delivery configuration, and wherein the tensioning member is configured to control expansion and compression of the second frame portion when not retained by the second housing.

EP38. The delivery catheter system according to any one of EP28 to EP37, comprising a balloon that is donut-shaped or lasso-shaped balloon configured and arranged to dilate the second frame portion in the expanded, deployed configuration, in use, to adjust or improve fixation, anchoring, placement, or sealing of the prosthesis.

EP39. The delivery catheter system according to any one of EP28 to EP38, wherein the tensioning member is configured to engage the second frame portion.

EP40. The delivery catheter system according to EP39, wherein the second frame portion comprises at least one retention member that the tensioning member is configured to engage.

EP41. The delivery catheter system according to EP40, wherein the retention member comprises an eyelet, loop, hook, ring, slit, slot, crown, or groove.

EP42. The delivery catheter system according to any one of EP28 to EP41, comprising a plurality of tensioning members and a plurality of retention members, wherein each tensioning member engages one or more different retention members.

EP43. The delivery catheter system according to EP42, wherein each of the plurality of tensioning members is independently actuatable to add or release tension.

EP44. The delivery catheter system according to any one of EP28 to EP43, wherein the tensioning member is actuatable to allow radial sections of the second frame portion to expand sequentially around a perimeter of the second frame portion.

EP45. The delivery catheter system according to any one of EP28 to EP44, wherein the catheter assembly comprises an atraumatic tip positioned at or near the end portion of the catheter body.

EP46. The delivery catheter system according to any one of EP28 to EP45, wherein the prosthesis is a valve prosthesis and comprises a valve body disposed within and secured to the first frame portion, the valve body being configured to prevent blood flow in one direction to convey blood flow through a central lumen of the first frame portion.

Although the teachings have been described above with reference to one or more preferred embodiments, it will be appreciated that various changes or modifications may be made without departing from the scope as defined in the appended claims.

What is claimed is:

1. A prosthesis having a compressed delivery configuration and an expanded, deployed configuration, the prosthesis comprising:
a frame comprising a first frame portion and a second frame portion; and
wherein the second frame portion is aligned longitudinally adjacent to the first frame portion when the prosthesis is in the compressed delivery configuration,
wherein the second frame portion is configured to anchor the prosthesis when the prosthesis is in the expanded deployed configuration, and
wherein when the prosthesis is in the expanded, deployed configuration the frame is everted such that the second frame portion is radially outward from the first frame portion.

2. The prosthesis according to claim 1, wherein, in the compressed delivery configuration, the first and second frame portions do not overlap along a longitudinal axis of the frame.

3. The prosthesis according to claim 1, wherein, in the compressed delivery configuration, the second frame portion is longitudinally offset from the first frame portion.

4. The prosthesis according to claim 1, wherein, in the deployed configuration, the second frame portion overlaps the first frame portion along a longitudinal axis of the frame.

5. The prosthesis according to claim 1, wherein, in the deployed configuration, the first and second frame portions are concentric and/or coaxial.

6. The prosthesis according to claim 1, wherein first frame portion is at least partially surrounded by the second frame portion when the prosthesis is in the expanded, deployed configuration.

7. The prosthesis according to claim 1, wherein, in the expanded, deployed configuration, the first frame portion defines a length that is greater than a length of the second frame portion along a longitudinal axis of the frame.

8. The prosthesis according to claim 1, wherein the frame comprises a plurality of cells arranged in a plurality of circumferential rows, and wherein the frame is configured such that the second frame portion comprises at least one row of cells.

9. The prosthesis according to claim 8, wherein each row comprises less than twelve cells, optionally less than ten cells, for example in the range four to ten cells, for example in the range six to nine cells.

10. The prosthesis according to claim 8, wherein each row comprises three, six, or nine cells.

11. The prosthesis according to claim 8, wherein at least one cell of the plurality of cells is substantially diamond-shaped.

12. The prosthesis according to claim 11, wherein each of the plurality of cells is substantially diamond-shaped.

13. The prosthesis according to claim 8, wherein the frame comprises a plurality of crowns and a plurality of struts with each crown being formed between a pair of opposing struts, and wherein the plurality of cells are defined by the plurality of crowns and the plurality of struts.

14. The prosthesis according to claim 1, wherein the second frame portion comprises at least one retention member configured for receiving a tensioning member to control eversion and/or deployment of the frame.

15. The delivery catheter system according to claim 14, wherein the retention member comprises an eyelet, loop, hook, ring, slit, slot, crown, or groove.

16. The prosthesis according to claim 14, wherein the frame comprises a series of end crowns at an end of the second frame portion, and wherein one or more of the end crowns comprise a retention member.

17. The prosthesis according to claim 1, wherein the frame is formed from a self-expanding material.

18. The prosthesis according to claim 17, wherein the self-expanding material comprises a shape-memory material, for example nitinol.

19. The prosthesis according to claim 17, wherein the self-expanding material is shape set to evert the second frame portion upon expansion of the prosthesis.

20. The prosthesis according to claim 17, wherein the second frame portion comprises at least one retention member, and wherein the self-expanding material is configured to expand radial sections of the second frame portion sequentially starting from a radial section corresponding to one group of at least one retention member and then proceeding to one or more other radial sections around a perimeter of the second frame portion.
